(19) United States
(12) Patent Application Publication
(10) Pub. No.: US 2010/0305068 A1
(43) Pub. Date: Dec. 2, 2010

(54) COMPOUNDS AND METHODS FOR MODULATING PROTEIN TRAFFICKING

(75) Inventors: Christine Bulawa, Arlington, MA (US); James Fleming, Somerville, MA (US)

Correspondence Address: FISH & RICHARDSON P.C., P.O. BOX 1022, MINNEAPOLIS, MN, 55440-1022, US

(73) Assignee: FoldRx Pharmaceuticals, Inc., Cambridge, MA (US)

(21) Appl. No.: 12/513,917

(22) PCT Filed: Nov. 9, 2007
(86) PCT No.: PCT/US07/84257

§ 371 Date: Aug. 17, 2010

Related U.S. Application Data
(60) Provisional application No. 60/857,941, filed on Nov. 9, 2006.

(51) Int. Cl. A61K 31/675

(52) U.S. Cl. 514/89

(57) ABSTRACT

Disclosed are compositions and methods for modulating protein trafficking and treating or preventing disorders characterized by impaired protein trafficking. Also disclosed are methods for producing a protein and identifying compounds that rescue protein trafficking defects.

### CROSS-REFERENCE TO RELATED APPLICATIONS

This application claims priority from U.S. Provisional Application No. 60/857,941, filed Nov. 9, 2006. The entire content of the prior application is incorporated herein by reference.

### TECHNICAL FIELD

This invention relates to compounds and methods for modulating protein trafficking and treating or preventing disorders characterized by impaired protein trafficking.

### BACKGROUND

Disorders characterized by impaired protein trafficking are numerous and include genetic diseases such as Huntington's disease, Tay-Sachs disease, familial hypercholesterolemia, and cystic fibrosis. Mutations in genes associated with these disorders often result in proteins that improperly fold and/or are retained in the endoplasmic reticulum. As a result, these proteins are often prematurely degraded.

The failure of a cell (e.g., in a tissue) to express a sufficient amount of an essential protein, e.g., an enzyme, can result in disease states, which vary in presentation and severity among protein trafficking disorders. For example, cystic fibrosis can affect nearly the entire body, causing progressive disability and early death. Difficulty breathing is the most common symptom and results from frequent lung infections, which can be treated by antibiotics and other medications. A multitude of other symptoms, including sinus infections, poor growth, diarrhea, and infertility can result from the effects of cystic fibrosis on other parts of the body. Cystic fibrosis, like many other disorders characterized by impaired protein trafficking, can be lethal if untreated.

Other protein trafficking disorders include, for example, synucleinopathies including but not limited to, Parkinson's disease, dementia with Lewy bodies, multiple system atrophy and the Lewy body variant of Alzheimer's disease.

For example, Parkinson's disease is a neurodegenerative disorder that is pathologically characterized by the presence of intracytoplasmic Lewy bodies (Lewy in Handbuch der Neurologie, M. Lewandowski, ed., Springer, Berlin, pp. 920-933, 1912; Pollanen et al., J. Neuropath. Exp. Neurol. 52:183-191, 1993), the major components of which are filaments consisting of α-synuclein (Spillantini et al., Proc. Natl. Acad. Sci. USA 95:6469-6473, 1998; Arai et al., Neurosci. Lett. 259:83-86, 1999), an 140-amino acid protein (Ueda et al., Proc. Natl. Acad. Sci. USA 90:11282-11286, 1993). Two dominant mutations in a-synuclein causing familial early onset Parkinson's disease have been described suggesting that Lewy bodies contribute mechanistically to the degeneration of neurons in Parkinson's disease and related disorders (Polymeropoulos et al., Science 276:2045-2047, 1997; Kruger et al., Nature Genet. 18:106-108, 1998; Zarranz et al., Ann. Neurol. 55:164-173, 2004). Triplication and duplication mutation of the a-synuclein gene have been linked to early-onset of Parkinson's disease (Singleton et al., Science 302:841, 2003; Chartier-Harlin at al. Lancet 364:1167-1169, 2004; Ibanez et al., Lancet 364:1169-1171, 2004). In vitro studies have demonstrated that recombinant α-synuclein can indeed form Lewy body-like fibrils (Conway et al., Nature Med. 4:1318-1320, 1998; Hashimoto et al., Brain Res. 799:301-306, 1998; Nahri et al., J. Biol. Chem. 274:9843-9846, 1999). Both Parkinson's disease-linked a-synuclein mutations accelerate this aggregation process, demonstrating that such in vitro studies may have relevance for Parkinson's disease pathogenesis. Alpha-synuclein aggregation and fibril formation fulfills of the criteria of a nucleation-dependent polymerization process (Wood et al., J. Biol. Chem. 274:19509-19512, 1999). In this regard a-synuclein fibril formation resembles that of Alzheimer's β-amyloid protein (Aβ) fibrils. Alpha-synuclein recombinant protein, and non-Aβ component (known as NAC), which is a 35-amino acid peptide fragment of α-synuclein, both have the ability to form fibrils when incubated at 37° C., and are positive with amyloid stains such as Congo red (demonstrating a red/green birefringence when viewed under polarized light) and Thioflavin S (demonstrating positive fluorescence) (Hashimoto et al., Brain Res. 799:301-306, 1998; Ueda et al., Proc. Natl. Acad. Sci. USA 90:11282-11286, 1993).

Fibrillization and aggregation of a-synuclein is thought to play major role in neuronal dysfunction and death of dopaminergic neurons in PD. Mutations in α-synuclein or genomic triplication of wild type α-synuclein (leading to its overexpression) cause certain rare familial forms of Parkinson's disease. In vitro and in vivo models suggest that over-expression of wild-type α-synuclein induces neuronal cell death. See, e.g., Polymeropoulos, et al. (1997) Science 276(5321):2045-7, Kruger, et al. (1998) Nat Genet. 18(2):106-8, Singleton, et al. (2003) Science 302(5646):841, Miller, et al. (2004) Neurology 62(10):1835-8, Hashimoto, et al. (2003) Ann NY Acad Sci. 991:171-88, Lo Bianco, et al. (2002) Proc Natl Acad Sci USA. 99(16):10813-8, Lee, et al. (2002) Proc Natl Acad Sci USA. 99(13):8968-73, Masliah, et al. (2000) Science 287(5456):1265-9, Auluck, et al. (2002) Science 295(5556):865-8, Oluwatosin-Chigbu et al. (2003) Biochem Biophys Res Commun 309(3): 679-84, Klucken et al. (2004) J Biol Chem. 279(24):25497-502. Protecting neurons from the toxic effects of α-synuclein is a promising strategy for treating Parkinson's disease and other synucleinopathies such as Lewy body dementia.

Thus, there is a need for compounds and compositions that rescue protein trafficking in order to treat diseases and disorders mediated by protein trafficking, such as cystic fibrosis and Parkinson's disease.

### SUMMARY

The invention is based, at least in part, on the identification of compounds that rescue protein trafficking defects. These compounds can be used to treat a variety of disorders characterized by impaired protein trafficking.

Described herein are methods of treating or preventing a disorder characterized by impaired protein trafficking by administering to a subject in need thereof an effective amount of a compound of Table 2 or a pharmaceutically acceptable derivative thereof or Table 3 or a pharmaceutically acceptable derivative thereof.

The disorder characterized by impaired protein trafficking can be a synucleinopathy. Examples of synucleinopathies include Parkinson's disease, Lewy body disease, the Lewy body variant of Alzheimer's disease, dementia with Lewy bodies, multiple system atrophy, or the Parkinsonism-dementia complex of Guam.

Synucleins are a family of small, presynaptic neuronal proteins composed of alpha-, beta-, and gamma-synucleins, of which only alpha-synuclein aggregates have been associated with several neurological diseases (Ian et al., Clinical Neurosc. Res. 1:445-455, 2001; Trojanowski and Lee, Neurotoxicology 23:457-460, 2002). The role of synucleins (and in particular, alpha-synuclein) in the etiology of a number of neurodegenerative and/or amyloid diseases has developed from several observations. Pathologically, alpha-synuclein was identified as a major component of Lewy bodies, the hallmark inclusions of Parkinson's disease, and a fragment thereof was isolated from amyloid plaques of a different neurological disease, Alzheimer's disease. Biochemically, recombinant alpha-synuclein was shown to form amyloid-like fibrils that recapitulated the ultrastructural features of alpha-synuclein isolated from patients with dementia with Lewy bodies, Parkinson's disease and multiple system atrophy. Additionally, the identification of mutations within the alpha-synuclein gene, albeit in rare cases of familial Parkinson's disease, demonstrated an unequivocal link between synuclein pathology and neurodegenerative diseases. The common involvement of alpha-synuclein in a spectrum of diseases such as Parkinson's disease, dementia with Lewy bodies, multiple system atrophy and the Lewy body variant of Alzheimer's disease has led to the classification of these diseases under the umbrella term of “synucleinopathies.”

In some embodiments, the disorder characterized by impaired protein trafficking is a lysosomal storage disorder such as Fabry disease, Farber disease, Gaucher disease, GM₁-gangliosidosis, Tay-Sachs disease, Sandhoff disease, GM₂ activator disease, Krabbe disease, metachromatic leukodystrophy, Niemann-Pick disease (types A, B, and C), Hurler disease, Scheie disease, Hunter disease, Sanfilippo disease, Morquio disease, Maroteaux-Lamy disease, hyaluronidase deficiency, aspartylglucosaminuria, fucosidosis, mannosidosis, Schindler disease, sialidosis type 1, Pompe disease, Pycnodysostosis, ceroid lipofuscinosis, cholesterol ester storage disease, Wolman disease, Multiple sulfatase, galactosialidosis, mucolipidosis (types II ,III, and IV), cystinosis, sialic acid storage disorder, chylomicron retention disease with Marinesco-Sjögren syndrome, Hermansky-Pudlak syndrome, Chediak-Higashi syndrome, Danon disease, or Geleophysic dysplasia. Lysosomal storage disorders are reviewed in, e.g., Wilcox (2004) J. Pediatr. 144:S3-S14.

In some embodiments, the disorder characterized by impaired protein trafficking is characterized by an impaired delivery of cargo to a cellular compartment.

In some embodiments, the disorder characterized by impaired protein trafficking is characterized by a Rab27a mutation or a deficiency of Rab27a. The disorder can be, e.g., Griscelli syndrome.

In some embodiments, the disorder characterized by impaired protein trafficking is cystic fibrosis.

In some embodiments, the disorder characterized by impaired protein trafficking is diabetes (e.g., diabetes mellitus).

In some embodiments, the disorder characterized by impaired protein trafficking is hereditary emphysema, α-1-antitrypsin deficiency, hereditary hemochromatosis, oculocutaneous albinism, protein C deficiency, type I hereditary angioedema, congenital sucrase-isomaltase deficiency, Crigler-Najjar type II, Laron syndrome, hereditary Myeloperoxidase, primary hypothyroidism, congenital long QT syndrome, tyroxine binding globulin deficiency, familial hypercholesterolemia, familial chylomicronemia, abeta-lipoproteinema, low plasma lipoprotein a levels, hereditary emphysema with liver injury, congenital hypothyroidism, osteogenesis imperfecta, hereditary hypofibrinogenemia, alpha-1antichymotrypsin deficiency, nephrogenic diabetes insipidus, neurohypophyseal diabetes, insipidus, Charcot-Marie-Tooth syndrome, Pelizaeus Merzbacher disease, von Willebrand disease type IIA, combined factors V and VIII deficiency, spondylo-epiphyseal dysplasia tarda, choroideremia, I cell disease, Batten disease, ataxia telangiectasias, acute lymphoblastic leukemia, acute myeloid leukemia, myeloid leukemia, ADPKD-autosomal dominant polycystic kidney disease, microvillus inclusion disease, tuberous sclerosis, oculocerebro-renal syndrome of Lowe, amyotrophic lateral sclerosis, myelodysplastic syndrome, Bare lymphocyte syndrome, Tangier disease, familial intrahepatic cholestasis, X-linked adreno-leukodystrophy, Scott syndrome, Hermansky-Pudlak syndrome types 1 and 2, Zellweger syndrome, rhizomelic chondrodysplasia puncta, autosomal recessive primary hyperoxaluria, Mohr Tranebjaerg syndrome, spinal and bullar muscular atrophy, primary ciliary diskenesia (Kartagener's syndrome), Miller Dieker syndrome, lissencephaly, motor neuron disease, Usher's syndrome, Wiskott-Aldrich syndrome, Optiz syndrome, Huntington's disease, hereditary pancreatitis, anti-phospholipid syndrome, overlap connective tissue disease, Sjögren's syndrome, stiff-man syndrome, Brugada syndrome, congenital nephritic syndrome of the Finnish type, Dubin-Johnson syndrome, X-linked hypophosphosphatemia, Pendred syndrome, persistent hyperinsulinemic hypoglycemia of infancy, hereditary spherocytosis, aceruloplasminemia, infantile neuronal ceroid lipofuscinosis, pseudoachondroplasia and multiple epiphyseal, Stargardt-like macular dystrophy, X-linked Charcot-Marie-Tooth disease, autosomal dominant retinitis pigmentosa, Wolcott-Rallison syndrome, Cushing's disease, limb-girdle muscular dystrophy, mucoploy-saccharidosis type IV, hereditary familial amyloidosis of Finish, Anderson disease, sarcoma, chronic myelomonocytic leukemia, cardiomyopathy, faciogenital dysplasia, Torsion disease, Huntington and spinocerebellar ataxias, hereditary hyperhomosyteinemia, polyneuropathy, lower motor neuron disease, pigmented retinitis, seronegative polyarthritis, interstitial pulmonary fibrosis, Raynaud's phenomenon, Wegner's granulomatosis, preoteinuria, CDG-Ia, CDG-Ib, CDG-Ic, CDG-Id, CDG-Ie, CDG-If, CDG-IIa, CDG-IIb, CDG-IIc, CDG-IId, Ehlers-Danlos syndrome, multiple exostoses, Griscelli syndrome (type 1 or type 2), or X-linked non-specific mental retardation. Disorders characterized by impaired protein trafficking are reviewed in Aridor et al. (2000) Traffic 1:836-51 and Aridor et al. (2002) Traffic 3:781-90.

Also disclosed is a composition comprising (i) a compound of Table 2 or a pharmaceutically acceptable derivative thereof or Table 3 or a pharmaceutically acceptable derivative thereof, and (ii) one or more of donepezil hydrochloride (Aracept), rivastigmine tartrate (Exelon), tacrine hydrochloride (Cognex), or galantamine hydrobromide (Reminyl).

Also disclosed are methods of treating or preventing a synucleinopathy by administering to a subject in need thereof an effective amount of a composition comprising (i) a compound of Table 2 or a pharmaceutically acceptable derivative thereof or Table 3 or a pharmaceutically acceptable derivative thereof, and (ii) one or more of donepezil hydrochloride (Aracept), rivastigmine tartrate (Exelon), tacrine hydrochloride (Cognex), or galantamine hydrobromide (Reminyl). The synucleinopathy can be, for example, Parkinson's disease, Lewy body disease, the Lewy body variant of Alzheimer's disease, dementia with Lewy bodies, multiple system atrophy, or the Parkinsonism-dementia complex of Guam.

The subject treated according to the methods described herein can be a human or another mammal such as a mouse, rat, cow, pig, dog, cat, or monkey.

Also disclosed are methods of inhibiting alpha synuclein-mediated cellular toxicity, the method comprising contacting a cell expressing a toxicity-inducing amount or form of alpha synuclein with an effective amount of a compound of Table 2 or a pharmaceutically acceptable derivative thereof or Table 3 or a pharmaceutically acceptable derivative thereof.

Also disclosed are methods of inhibiting alpha synuclein-mediated cellular toxicity, the method comprising contacting a cell expressing a toxicity-inducing amount or form of alpha synuclein with an effective amount of a composition comprising (i) a compound of Table 2 or a pharmaceutically acceptable derivative thereof or Table 3 or a pharmaceutically acceptable derivative thereof, and (ii) one or more of donepezil hydrochloride (Aracept), rivastigmine tartrate (Exelon), tacrine hydrochloride (Cognex), or galantamine hydrobromide (Reminyl).

In addition to the compounds of Table 2 and Table 3, pharmaceutically-acceptable derivatives (including salts, esters, enol ethers, enol esters, solvates, hydrates and prodrugs) of the compounds can be used in the methods described herein. Pharmaceutically-acceptable salts, include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc, aluminum, and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates.

Further provided are pharmaceutical compositions containing the compounds provided herein and a pharmaceutically acceptable carrier. The pharmaceutical compositions can optionally be formulated for single dosage administration. In practicing the methods, effective amounts of the compounds or compositions containing therapeutically effective concentrations of the compounds are administered.

Articles of manufacture are provided containing packaging material, a compound or composition described herein, and a label that indicates that the compound or composition is useful for treating or ameliorating one or more symptoms of a disorder characterized by impaired protein trafficking (e.g., a synucleinopathy such as Parkinson's disease).

Also disclosed is a method of producing a protein, which method includes the steps of: culturing a cell in the presence of a compound described herein (e.g., a compound depicted in Table 2 or a pharmaceutically acceptable derivative thereof or a compound depicted in Table 3 or a pharmaceutically acceptable derivative thereof); and purifying a protein produced by the cell, wherein the culturing of the cell in the presence of the compound results in enhanced production of the purified protein as compared to culture of the cell in the absence of the compound. The protein can be a recombinant protein encoded by a heterologous nucleic acid. In some embodiments, the protein is a secreted protein and/or a glycosylated protein. For example, the protein can be a cytokine, a lymphokine, a growth factor, or an antibody. The cell used in the protein production methods can be, e.g., an insect cell, a mammalian cell (e.g., a Chinese Hamster Ovary cell), a fungal cell, or a bacterial cell.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the preferred methods and materials are described below. All publications, patent applications, patents, and other references mentioned herein are incorporated by reference in their entirety. In case of conflict, the present application, including definitions, will control. In addition, the materials, methods, and examples are illustrative only and not intended to be limiting.

Other features and advantages of the invention will be apparent from the following detailed description, and from the claims.

### DETAILED DESCRIPTION OF THE INVENTION

### A. Definitions

As used herein, alpha-synuclein refers to one in a family of structurally related proteins that are prominently expressed in the central nervous system. Aggregated alpha-synuclein proteins form brain lesions that are hallmarks of some neurodegenerative diseases (synucleinopathies). The gene for alpha-synuclein, which is called SNCA, is on chromosome 4q21. One form of hereditary Parkinson disease is due to mutations in SNCA. Another form of hereditary Parkinson disease is due to a triplication of SNCA.

As used herein, pharmaceutically acceptable derivatives of a compound include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, nitrates, borates, methanesulfonates, benzenesulfonates, toluenesulfonates, salts of mineral acids, such as but not limited to hydrochlorides, hydrobromides, hydroiodides and sulfates; and salts of organic acids, such as but not limited to acetates, trifluoroacetates, maleates, oxalates, lactates, malates, tartrates, citrates, benzoates, salicylates, ascorbates, succinates, butyrates, valerates and fumarates. Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, aryl, heteroaryl, aralkyl, heteroaralkyl, cycloalkyl or heterocyclyl. Pharmaceutically acceptable enol esters include, but are not limited to, derivatives of formula C═C(OC(O)R) where R is hydrogen, alkyl, alkenyl, alkynyl, aryl, heteroaryl, aralkyl, heteroaralkyl, cycloalkyl or heterocyclyl. Pharmaceutically acceptable solvates and hydrates are complexes of a compound with one or more solvent or water molecules, or 1 to about 100, or 1 to about 10, or one to about 2, 3 or 4, solvent or water molecules.

As used herein, treatment means any manner in which one or more of the symptoms of a disease or disorder are ameliorated or otherwise beneficially altered. As used herein, amelioration of the symptoms of a particular disorder by administration of a particular compound or pharmaceutical composition refers to any lessening, whether permanent or temporary, lasting or transient that can be attributed to or associated with administration of the composition.

As used herein, IC₅₀ refers to an amount, concentration or dosage of a particular test compound that achieves a 50% inhibition of a maximal response, such as modulation of protein trafficking, in an assay that measures such response.

As used herein, EC₅₀ refers to a dosage, concentration or amount of a particular test compound that elicits a dose-dependent response at 50% of maximal expression of a particular response that is induced, provoked or potentiated by the particular test compound.

As used herein, a prodrug is a compound that, upon in vivo administration, is metabolized by one or more steps or processes or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

It is to be understood that the compounds provided herein may contain chiral centers. Such chiral centers may be of either the (R) or (S) configuration, or may be a mixture thereof Thus, the compounds provided herein may be enantiomerically pure, or be stereoisomeric or diastereomeric mixtures. In the case of amino acid residues, such residues may be of either the L- or D-form. The configuration for naturally occurring amino acid residues is generally L. When not specified the residue is the L form. As used herein, the term “amino acid” refers to a-amino acids which are racemic, or of either the D- or L-configuration. The designation “d” preceding an amino acid designation (e.g., dAla, dSer, dVal, etc.) refers to the D-isomer of the amino acid. The designation “dl” preceding an amino acid designation (e.g., dlPip) refers to a mixture of the L- and D-isomers of the amino acid. It is to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

As used herein, substantially pure means sufficiently homogeneous to appear free of readily detectable impurities as determined by standard methods of analysis, such as thin layer chromatography (TLC), gel electrophoresis, high performance liquid chromatography (HPLC) and mass spectrometry (MS), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers. In such instances, further purification might increase the specific activity of the compound.

As used herein, “alkyl,” “alkenyl” and “alkynyl” carbon chains, if not specified, contain from 1 to 20 carbons, or 1 or 2 to 16 carbons, and are straight or branched. Alkenyl carbon chains of from 2 to 20 carbons, in certain embodiments, contain 1 to 8 double bonds and alkenyl carbon chains of 2 to 16 carbons, in certain embodiments, contain 1 to 5 double bonds. Alkynyl carbon chains of from 2 to 20 carbons, in certain embodiments, contain 1 to 8 triple bonds, and the alkynyl carbon chains of 2 to 16 carbons, in certain embodiments, contain 1 to 5 triple bonds. Exemplary alkyl, alkenyl and alkynyl groups herein include, but are not limited to, methyl, ethyl, propyl, isopropyl, isobutyl, n-butyl, sec-butyl, tert-butyl, isopentyl, neopentyl, tert-pentyl, isohexyl, allyl (propenyl) and propargyl (propynyl). As used herein, lower alkyl, lower alkenyl, and lower alkynyl refer to carbon chains having from about 1 or about 2 carbons up to about 6 carbons. As used herein, “alk(en)(yn)yl” refers to an alkyl group containing at least one double bond and at least one triple bond.

As used herein, “cycloalkyl” refers to a saturated mono- or multi-cyclic ring system, in certain embodiments of 3 to 10 carbon atoms, in other embodiments of 3 to 6 carbon atoms; cycloalkenyl and cycloalkynyl refer to mono- or multicyclic ring systems that respectively include at least one double bond and at least one triple bond. Cycloalkenyl and cycloalkynyl groups may, in certain embodiments, contain 3 to 10 carbon atoms, with cycloalkenyl groups, in further embodiments, containing 4 to 7 carbon atoms and cycloalkynyl groups, in further embodiments, containing 8 to 10 carbon atoms. The ring systems of the cycloalkyl, cycloalkenyl and cycloalkynyl groups may be composed of one ring or two or more rings which may be joined together in a fused, bridged or spiro-connected fashion. “Cycloalk(en)(yn)yl” refers to a cycloalkyl group containing at least one double bond and at least one triple bond.

As used herein, “aryl” refers to aromatic monocyclic or multicyclic groups containing from 6 to 19 carbon atoms. Aryl groups include, but are not limited to groups such as unsubstituted or substituted fluorenyl, unsubstituted or substituted phenyl, and unsubstituted or substituted naphthyl.

As used herein, “heteroaryl” refers to a monocyclic or multicyclic aromatic ring system, in certain embodiments, of about 5 to about 15 members where one or more, in one embodiment 1 to 3, of the atoms in the ring system is a heteroatom, that is, an element other than carbon, including but not limited to, nitrogen, oxygen or sulfur. The heteroaryl group may be optionally fused to a benzene ring. Heteroaryl groups include, but are not limited to, furyl, imidazolyl, pyrimidinyl, tetrazolyl, thienyl, pyridyl, pyrrolyl, thiazolyl, isothiazolyl, oxazolyl, isoxazolyl, triazolyl, quinolinyl and isoquinolinyl.

As used herein, a “heteroarylium” group is a heteroaryl group that is positively charged on one or more of the heteroatoms.

As used herein, “heterocyclyl” refers to a monocyclic or multicyclic non-aromatic ring system, in one embodiment of 3 to 10 members, in another embodiment of 4 to 7 members, in a further embodiment of 5 to 6 members, where one or more, in certain embodiments, 1 to 3, of the atoms in the ring system is a heteroatom, that is, an element other than carbon, including but not limited to, nitrogen, oxygen or sulfur. In embodiments where the heteroatom(s) is(are) nitrogen, the nitrogen is optionally substituted with alkyl, alkenyl, alkynyl, aryl, heteroaryl, aralkyl, heteroaralkyl, cycloalkyl, heterocyclyl, cycloalkylalkyl, heterocyclylalkyl, acyl, guanidino, or the nitrogen may be quaternized to form an ammonium group where the substituents are selected as above.

As used herein, “aralkyl” refers to an alkyl group in which one of the hydrogen atoms of the alkyl is replaced by an aryl group.

As used herein, “heteroaralkyl” refers to an alkyl group in which one of the hydrogen atoms of the alkyl is replaced by a heteroaryl group.

As used herein, “halo”, “halogen” or “halide” refers to F, Cl, Br or I.

As used herein, pseudohalides or pseudohalo groups are groups that behave substantially similar to halides. Such compounds can be used in the same manner and treated in the same manner as halides. Pseudohalides include, but are not limited to, cyanide, cyanate, thiocyanate, selenocyanate, trifluoromethoxy, and azide.

As used herein, “haloalkyl” refers to an alkyl group in which one or more of the hydrogen atoms are replaced by halogen. Such groups include, but are not limited to, chloromethyl, trifluoromethyl and1-chloro-2-fluoroethyl.

As used herein, “haloalkoxy” refers to RO— in which R is a haloalkyl group.

As used herein, “sulfinyl” or “thienyl” refers to —S(O)—. As used herein, “sulfonyl” or “sulfuryl” refers to —S(O)₂—. As used herein, “sulfo” refers to —S(O)₂O—.

As used herein, “carboxy” refers to a divalent radical, —C(O)O—.

As used herein, “aminocarbonyl” refers to —C(O)NH₂.

As used herein, “alkylaminocarbonyl” refers to —C(O)NHR in which R is alkyl, including lower alkyl. As used herein, “dialkylaminocarbonyl” refers to —C(O)NR′R in which R′ and R are independently alkyl, including lower alkyl; “carboxamide” refers to groups of formula —NR′COR in which R′ and R are independently alkyl, including lower alkyl.

As used herein, “diarylaminocarbonyl” refers to —C(O)NRR' in which R and R′ are independently selected from aryl, including lower aryl, such as phenyl.

As used herein, “arylalkylaminocarbonyl” refers to —C(O)NRR′ in which one of R and R′ is aryl, including lower aryl, such as phenyl, and the other of R and R′ is alkyl, including lower alkyl.

As used herein, “arylaminocarbonyl” refers to —C(O)NHR in which R is aryl, including lower aryl, such as phenyl.

As used herein, “hydroxycarbonyl” refers to —COOH.

As used herein, “alkoxycarbonyl” refers to —C(O)OR in which R is alkyl, including lower alkyl.

As used herein, “aryloxycarbonyl” refers to —C(O)OR in which R is aryl, including lower aryl, such as phenyl.

As used herein, “alkoxy” and “alkylthio” refer to RO— and RS—, in which R is alkyl, including lower alkyl.

As used herein, “aryloxy” and “arylthio” refer to RO— and RS—, in which R is aryl, including lower aryl, such as phenyl.

As used herein, “alkylene” refers to a straight, branched or cyclic, in certain embodiments straight or branched, divalent aliphatic hydrocarbon group, in one embodiment having from 1 to about 20 carbon atoms, in another embodiment having from 1 to 12 carbons. In a further embodiment alkylene includes lower alkylene. There may be optionally inserted along the alkylene group one or more oxygen, sulfur, including S(═O) and S(═O)₂ groups, or substituted or unsubstituted nitrogen atoms, including —NR— and —N⁺RR— groups, where the nitrogen substituent(s) is(are) alkyl, aryl, aralkyl, heteroaryl, heteroaralkyl or COR′, where R′ is alkyl, aryl, aralkyl, heteroaryl, heteroaralkyl, —OY or —NYY, where Y is hydrogen, alkyl, aryl, heteroaryl, cycloalkyl or heterocyclyl. Alkylene groups include, but are not limited to, methylene (—CH₂—), ethylene (—CH₂CH₂—), propylene (—(CH₂)₃—), methylenedioxy (—O—CH₂—O—) and ethylenedioxy (—O—(CH₂)₂—O—). The term “lower alkylene” refers to alkylene groups having 1 to 6 carbons. In certain embodiments, alkylene groups are lower alkylene, including alkylene of 1 to 3 carbon atoms.

As used herein, “azaalkylene” refers to —(CRR)ₙ—NR—(CRR)ₘ—, where n and m are each independently an integer from 0 to 4. As used herein,“oxaalkylene” refers to —(CRR)ₙ—O—(CRR)ₘ—, where n and m are each independently an integer from 0 to 4. As used herein, “thiaalkylene” refers to —(CRR)ₙ—S—(CRR)ₘ—, —(CRR)ₙ—S(═O)—(CRR)ₘ—, and —(CRR)ₙ—S(═O)₂—(CRR)ₘ—, where n and m are each independently an integer from 0 to 4.

As used herein, “alkenylene” refers to a straight, branched or cyclic, in one embodiment straight or branched, divalent aliphatic hydrocarbon group, in certain embodiments having from 2 to about 20 carbon atoms and at least one double bond, in other embodiments 1 to 12 carbons. In further embodiments, alkenylene groups include lower alkenylene. There may be optionally inserted along the alkenylene group one or more oxygen, sulfur or substituted or unsubstituted nitrogen atoms, where the nitrogen substituent is alkyl. Alkenylene groups include, but are not limited to, —CH═CH—CH═CH— and —CH═CH—CH₂—. The term “lower alkenylene” refers to alkenylene groups having 2 to 6 carbons. In certain embodiments, alkenylene groups are lower alkenylene, including alkenylene of 3 to 4 carbon atoms.

As used herein, “alkynylene” refers to a straight, branched or cyclic, in certain embodiments straight or branched, divalent aliphatic hydrocarbon group, in one embodiment having from 2 to about 20 carbon atoms and at least one triple bond, in another embodiment 1 to 12 carbons. In a further embodiment, alkynylene includes lower alkynylene. There may be optionally inserted along the alkynylene group one or more oxygen, sulfur or substituted or unsubstituted nitrogen atoms, where the nitrogen substituent is alkyl. Alkynylene groups include, but are not limited to, —C≡C—C≡C—, —C≡C— and —C≡C—CH₂—. The term “lower alkynylene” refers to alkynylene groups having 2 to 6 carbons. In certain embodiments, alkynylene groups are lower alkynylene, including alkynylene of 3 to 4 carbon atoms.

As used herein, “alk(en)(yn)ylene” refers to a straight, branched or cyclic, in certain embodiments straight or branched, divalent aliphatic hydrocarbon group, in one embodiment having from 2 to about 20 carbon atoms and at least one triple bond, and at least one double bond; in another embodiment 1 to 12 carbons. In further embodiments, alk(en)(yn)ylene includes lower alk(en)(yn)ylene. There may be optionally inserted along the alkynylene group one or more oxygen, sulfur orsubstituted or unsubstituted nitrogen atoms, where the nitrogen substituent is alkyl. Alk(en)(yn)ylene groups include, but are not limited to, —C═C—(CH₂)ₙ—C≡C—, where n is 1 or 2. The term “lower alk(en)(yn)ylene” refers to alk(en)(yn)ylene groups having up to 6 carbons. In certain embodiments, alk(en)(yn)ylene groups have about 4 carbon atoms.

As used herein, “cycloalkylene” refers to a divalent saturated mono- or multicyclic ring system, in certain embodiments of 3 to 10 carbon atoms, in other embodiments 3 to 6 carbon atoms; cycloalkenylene and cycloalkynylene refer to divalent mono- or multicyclic ring systems that respectively include at least one double bond and at least one triple bond. Cycloalkenylene and cycloalkynylene groups may, in certain embodiments, contain 3 to 10 carbon atoms, with cycloalkenylene groups in certain embodiments containing 4 to 7 carbon atoms and cycloalkynylene groups in certain embodiments containing 8 to 10 carbon atoms. The ring systems of the cycloalkylene, cycloalkenylene and cycloalkynylene groups may be composed of one ring or two or more rings which may be joined together in a fused, bridged or spiro-connected fashion. “Cycloalk(en)(yn)ylene” refers to a cycloalkylene group containing at least one double bond and at least one triple bond.

As used herein, “arylene” refers to a monocyclic or polycyclic, in certain embodiments monocyclic, divalent aromatic group, in one embodiment having from 5 to about 20 carbon atoms and at least one aromatic ring, in another embodiment 5 to 12 carbons. In further embodiments, arylene includes lower arylene. Arylene groups include, but are not limited to, 1,2-, 1,3- and 1,4-phenylene. The term “lower arylene” refers to arylene groups having 6 carbons.

As used herein, “heteroarylene” refers to a divalent monocyclic or multicyclic aromatic ring system, in one embodiment of about 5 to about 15 atoms in the ring(s), where one or more, in certain embodiments 1 to 3, of the atoms in the ring system is a heteroatom, that is, an element other than carbon, including but not limited to, nitrogen, oxygen or sulfur. The term “lower heteroarylene” refers to heteroarylene groups having 5 or 6 atoms in the ring.

As used herein, “heterocyclylene” refers to a divalent monocyclic or multicyclic non-aromatic ring system, in certain embodiments of 3 to 10 members, in one embodiment 4 to 7 members, in another embodiment 5 to 6 members, where one or more, including 1 to 3, of the atoms in the ring system is a heteroatom, that is, an element other than carbon, including but not limited to, nitrogen, oxygen or sulfur.

As used herein, “substituted alkyl,” “substituted alkenyl,” “substituted alkynyl,” “substituted cycloalkyl,” “substituted cycloalkenyl,” “substituted cycloalkynyl,” “substituted aryl,” “substituted heteroaryl,” “substituted heterocyclyl,” “substituted alkylene,” “substituted alkenylene,” “substituted alkynylene,” “substituted cycloalkylene,” “substituted cycloalkenylene,” “substituted cycloalkynylene,” “substituted arylene,” “substituted heteroarylene” and “substituted heterocyclylene” refer to alkyl, alkenyl, alkynyl, cycloalkyl, cycloalkenyl, cycloalkynyl, aryl, heteroaryl, heterocyclyl, alkylene, alkenylene, alkynylene, cycloalkylene, cycloalkenylene, cycloalkynylene, arylene, heteroarylene and heterocyclylene groups, respectively, that are substituted with one or more substituents, in certain embodiments one, two, three or four substituents, where the substituents are as defined herein.

As used herein, “alkylidene” refers to a divalent group, such as ═CR′R″, which is attached to one atom of another group, forming a double bond. Alkylidene groups include, but are not limited to, methylidene (═CH₂) and ethylidene (═CHCH₃). As used herein, “arylalkylidene” refers to an alkylidene group in which either R′ or R″ is an aryl group. “Cycloalkylidene” groups are those where R′ and R″ are linked to form a carbocyclic ring. “Heterocyclylid-ene” groups are those where at least one of R′ and R″ contain a heteroatom in the chain, and R′ and R″ are linked to form a heterocyclic ring.

As used herein, “amido” refers to the divalent group —C(O)NH—. “Thioamido” refers to the divalent group —C(S)NH—. “Oxyamido” refers to the divalent group —OC(O)NH—. “Thiaamido” refers to the divalent group —SC(O)NH—. “Dithiaamido” refers to the divalent group —SC(S)NH—. “Ureido” refers to the divalent group —HNC(O)NH—. “Thioureido” refers to the divalent group —HNC(S)NH—.

As used herein, “semicarbazide” refers to —NHC(O)NHNH—. “Carbazate” refers to the divalent group —OC(O)NHNH—. “Isothiocarbazate” refers to the divalent group —SC(O)NHNH—. “Thiocarbazate” refers to the divalent group —OC(S)NHNH—. “Sulfonylhydrazide” refers to the divalent group —SO₂NHNH—. “Hydrazide” refers to the divalent group —C(O)NHNH—. “Azo” refers to the divalent group —N═N—. “Hydrazinyl” refers to the divalent group —NH—NH—.

Where the number of any given substituent is not specified (e.g., haloalkyl), there may be one or more substituents present. For example, “haloalkyl” may include one or more of the same or different halogens.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:942-944).

### B. Compounds

The compounds disclosed in Table 2 and Table 3 and pharmaceutically acceptable derivatives thereof can be used in the compositions and methods provided herein to rescue protein trafficking defects and treat or prevent a wide variety of disorders characterized by impaired protein trafficking.

### C. Preparation of the Compounds

The compounds for use in the compositions and methods provided herein may be obtained from commercial sources (e.g., Aldrich Chemical Co., Milwaukee, Wis.) and may be prepared by methods well known to those of skill in the art. The compounds of Table 2 and Table 3 were obtained by screening of a commercial library (“The Spectrum Collection”) obtained from Microsource Discovery Systems, Inc. (Gaylordsville, Conn.).

### D. Formulation of Pharmaceutical Compositions

The pharmaceutical compositions provided herein contain therapeutically effective amounts of one or more of the compounds provided herein that are useful in the treatment or amelioration of one or more of the symptoms of diseases or disorders characterized by impaired protein trafficking, and a pharmaceutically acceptable carrier. Pharmaceutical carriers suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients.

The compositions contain one or more compounds provided herein. The compounds are, in one embodiment, formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In one embodiment, the compounds described above are formulated into pharmaceutical compositions using techniques and procedures well known in the art (see, e.g., Ansel Introduction to Pharmaceutical Dosage Forms, Fourth Edition 1985, 126).

In the compositions, effective concentrations of one or more compounds or pharmaceutically acceptable derivatives thereof is (are) mixed with a suitable pharmaceutical carrier. The compounds may be derivatized as the corresponding salts, esters, enol ethers or esters, acetals, ketals, orthoesters, hemiacetals, hemiketals, acids, bases, solvates, hydrates or prodrugs prior to formulation, as described above. The concentrations of the compounds in the compositions are effective for delivery of an amount, upon administration, that treats or ameliorates one or more of the symptoms of diseases or disorders characterized by impaired protein trafficking.

In one embodiment, the compositions are formulated for single dosage administration. To formulate a composition, the weight fraction of compound is dissolved, suspended, dispersed or otherwise mixed in a selected carrier at an effective concentration such that the treated condition is relieved or one or more symptoms are ameliorated.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in systems described herein (see Examples), and then extrapolated therefrom for dosages for humans.

The concentration of active compound in the pharmaceutical composition will depend on absorption, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of diseases or disorders characterized by impaired protein trafficking, as described herein.

In one embodiment, a therapeutically effective dosage should produce a serum concentration of active ingredient of from about 0.1 ng/ml to about 50-100 μg/ml. The pharmaceutical compositions, in another embodiment, should provide a dosage of from about 0.001 mg to about 2000 mg of compound per kilogram of body weight per day. Pharmaceutical dosage unit forms are prepared to provide from about 0.01 mg, 0.1 mg or 1 mg to about 500 mg, 1000 mg or 2000 mg, and in one embodiment from about 10 mg to about 500 mg of the active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate. Derivatives of the compounds, such as prodrugs of the compounds may also be used in formulating effective pharmaceutical compositions.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are, in one embodiment, formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrine derivatives, sorbitan monolaurate, triethanolamine sodium acetate, triethanolamine oleate, and other such agents.

Actual methods of preparing such dosage forms are known, or will be apparent, to those skilled in this art; for example, see Remington's Pharmaceutical Sciences, Mack Publishing Company, Easton, Pa., 15th Edition, 1975.

Dosage forms or compositions containing active ingredient in the range of 0.005% to 100% with the balance made up from non-toxic carrier may be prepared. Methods for preparation of these compositions are known to those skilled in the art. The contemplated compositions may contain 0.001%-100% active ingredient, in one embodiment 0.1-95%, in another embodiment 75-85%.

1. Compositions for Oral Administration

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

a. Solid Compositions for Oral Administration

In certain embodiments, the formulations are solid dosage forms, in one embodiment, capsules or tablets. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polvinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

The compound, or pharmaceutically acceptable derivative thereof, could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable derivative thereof as described herein. Higher concentrations, up to about 98% by weight of the active ingredient may be included.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

b. Liquid Compositions for Oral Administration

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic acid, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is in one embodiment encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. RE28,819 and 4,358,603. Briefly, such formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

2. Injectables, Solutions and Emulsions

Parenteral administration, in one embodiment characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow-release or sustained-release system, such that a constant level of dosage is maintained (see, e.g., U.S. Pat. No. 3,710,795) is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations are packaged in an ampoule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. In one embodiment, a therapeutically effective dosage is formulated to contain a concentration of at least about 0.1% w/w up to about 90% w/w or more, in certain embodiments more than 1% w/w of the active compound to the treated tissue(s).

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

3. Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

4. Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will, in one embodiment, have diameters of less than 50 microns, in one embodiment less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

5. Compositions for Other Routes of Administration

Other routes of administration, such as transdermal patches, including iontophoretic and electrophoretic devices, and rectal administration, are also contemplated herein.

Transdermal patches, including iotophoretic and electrophoretic devices, are well known to those of skill in the art. For example, such patches are disclosed in U.S. Pat. Nos. 6,267,983, 6,261,595, 6,256,533, 6,167,301, 6,024,975, 6,010715, 5,985,317, 5,983,134, 5,948,433, and 5,860,957.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The weight of a rectal suppository, in one embodiment, is about 2 to 3 gm.

Tablets and capsules for rectal administration are manufactured using the same pharmaceutically acceptable substance and by the same methods as for formulations for oral administration.

6. Targeted Formulations

The compounds provided herein, or pharmaceutically acceptable derivatives thereof, may also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

7. Articles of Manufacture

The compounds or pharmaceutically acceptable derivatives may be packaged as articles of manufacture containing packaging material, a compound or pharmaceutically acceptable derivative thereof provided herein, which is effective for treatment or amelioration of one or more symptoms of diseases or disorders characterized by impaired protein trafficking, within the packaging material, and a label that indicates that the compound or composition, or pharmaceutically acceptable derivative thereof, is used for treatment or amelioration of one or more symptoms of diseases or disorders characterized by impaired protein trafficking.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any disease or disorder in which impaired protein trafficking is implicated as a mediator or contributor to the symptoms or cause.

8. Sustained Release Formulations

Also provided are sustained release formulations to deliver the compounds to the desired target (i.e. brain or systemic organs) at high circulating levels (between 10⁻⁹ and 10⁻⁴ M). In a certain embodiment for the treatment of a disorder characterized by impaired protein trafficking, the circulating levels of the compounds is maintained up to 10⁻⁷M.

It is understood that the compound levels are maintained over a certain period of time as is desired and can be easily determined by one skilled in the art. In one embodiment, the administration of a sustained release formulation is effected so that a constant level of therapeutic compound is maintained between 10⁻⁸ and 10⁻⁶M between 48 to 96 hours in the sera.

Such sustained and/or timed release formulations may be made by sustained release means of delivery devices that are well known to those of ordinary skill in the art, such as those described in U.S. Pat. Nos. 3,845,770; 3,916,899; 3,536,809; 3,598,123; 4,008,719; 4,710,384; 5,674,533; 5,059,595; 5,591,767; 5,120,548; 5,073,543; 5,639,476; 5,354,556 and 5,733,566, the disclosures of which are each incorporated herein by reference. These pharmaceutical compositions can be used to provide slow or sustained release of one or more of the active compounds using, for example, hydroxypropylmethyl cellulose, other polymer matrices, gels, permeable membranes, osmotic systems, multilayer coatings, microparticles, liposomes, microspheres, or the like. Suitable sustained release formulations known to those skilled in the art, including those described herein, may be readily selected for use with the pharmaceutical compositions provided herein. Thus, single unit dosage forms suitable for oral administration, such as, but not limited to, tablets, capsules, gelcaps, caplets, powders and the like, that are adapted for sustained release are contemplated herein.

In one embodiment, the sustained release formulation contains active compound such as, but not limited to, microcrystalline cellulose, maltodextrin, ethylcellulose, and magnesium stearate. As described above, all known methods for encapsulation which are compatible with properties of the disclosed compounds are contemplated herein. The sustained release formulation is encapsulated by coating particles or granules of the pharmaceutical compositions provided herein with varying thickness of slowly soluble polymers or by microencapsulation. In one embodiment, the sustained release formulation is encapsulated with a coating material of varying thickness (e.g. about 1 micron to 200 microns) that allow the dissolution of the pharmaceutical composition about 48 hours to about 72 hours after administration to a mammal. In another embodiment, the coating material is a food-approved additive.

In another embodiment, the sustained release formulation is a matrix dissolution device that is prepared by compressing the drug with a slowly soluble polymer carrier into a tablet. In one embodiment, the coated particles have a size range between about 0.1 to about 300 microns, as disclosed in U.S. Pat. Nos. 4,710,384 and 5,354,556, which are incorporated herein by reference in their entireties. Each of the particles is in the form of a micromatrix, with the active ingredient uniformly distributed throughout the polymer.

Sustained release formulations such as those described in U.S. Pat. No. 4,710,384, which is incorporated herein by reference in its entirety, having a relatively high percentage of plasticizer in the coating in order to permit sufficient flexibility to prevent substantial breakage during compression are disclosed. The specific amount of plasticizer varies depending on the nature of the coating and the particular plasticizer used. The amount may be readily determined empirically by testing the release characteristics of the tablets formed. If the medicament is released too quickly, then more plasticizer is used. Release characteristics are also a function of the thickness of the coating. When substantial amounts of plasticizer are used, the sustained release capacity of the coating diminishes. Thus, the thickness of the coating may be increased slightly to make up for an increase in the amount of plasticizer. Generally, the plasticizer in such an embodiment will be present in an amount of about 15 to 30% of the sustained release material in the coating, in one embodiment 20 to 25%, and the amount of coating will be from 10 to 25% of the weight of the active material, and in another embodiment, 15 to 20% of the weight of active material. Any conventional pharmaceutically acceptable plasticizer may be incorporated into the coating.

The compounds provided herein can be formulated as a sustained and/or timed release formulation. All sustained release pharmaceutical products have a common goal of improving drug therapy over that achieved by their non-sustained counterparts. Ideally, the use of an optimally designed sustained release preparation in medical treatment is characterized by a minimum of drug substance being employed to cure or control the condition. Advantages of sustained release formulations may include: 1) extended activity of the composition, 2) reduced dosage frequency, and 3) increased patient compliance. In addition, sustained release formulations can be used to affect the time of onset of action or other characteristics, such as blood levels of the composition, and thus can affect the occurrence of side effects.

The sustained release formulations provided herein are designed to initially release an amount of the therapeutic composition that promptly produces the desired therapeutic effect, and gradually and continually release of other amounts of compositions to maintain this level of therapeutic effect over an extended period of time. In order to maintain this constant level in the body, the therapeutic composition must be released from the dosage form at a rate that will replace the composition being metabolized and excreted from the body.

The sustained release of an active ingredient may be stimulated by various inducers, for example pH, temperature, enzymes, water, or other physiological conditions or compounds.

Preparations for oral administration may be suitably formulated to give controlled release of the active compound. In one embodiment, the compounds are formulated as controlled release powders of discrete microparticles that can be readily formulated in liquid form. The sustained release powder comprises particles containing an active ingredient and optionally, an excipient with at least one non-toxic polymer.

The powder can be dispersed or suspended in a liquid vehicle and will maintain its sustained release characteristics for a useful period of time. These dispersions or suspensions have both chemical stability and stability in terms of dissolution rate. The powder may contain an excipient comprising a polymer, which may be soluble, insoluble, permeable, impermeable, or biodegradable. The polymers may be polymers or copolymers. The polymer may be a natural or synthetic polymer. Natural polymers include polypeptides (e.g., zein), polysaccharides (e.g., cellulose), and alginic acid. Representative synthetic polymers include those described, but not limited to, those described in column 3, lines 33-45 of U.S. Pat. No. 5,354,556, which is incorporated by reference in its entirety. Particularly suitable polymers include those described, but not limited to those described in column 3, line 46-column 4, line 8 of U.S. Pat. No. 5,354,556 which is incorporated by reference in its entirety.

The sustained release compositions provided herein may be formulated for parenteral administration, e.g., by intramuscular injections or implants for subcutaneous tissues and various body cavities and transdermal devices. In one embodiment, intramuscular injections are formulated as aqueous or oil suspensions. In an aqueous suspension, the sustained release effect is due to, in part, a reduction in solubility of the active compound upon complexation or a decrease in dissolution rate. A similar approach is taken with oil suspensions and solutions, wherein the release rate of an active compound is determined by partitioning of the active compound out of the oil into the surrounding aqueous medium. Only active compounds which are oil soluble and have the desired partition characteristics are suitable. Oils that may be used for intramuscular injection include, but are not limited to, sesame, olive, arachis, maize, almond, soybean, cottonseed and castor oil.

A highly developed form of drug delivery that imparts sustained release over periods of time ranging from days to years is to implant a drug-bearing polymeric device subcutaneously or in various body cavities. The polymer material used in an implant, which must be biocompatible and nontoxic, include but are not limited to hydrogels, silicones, polyethylenes, ethylene-vinyl acetate copolymers, or biodegradable polymers.

E. Evaluation of the Activity of the Compounds

The activity of the compounds as modulators of protein trafficking may be measured in the assays described herein that evaluate the ability of a compound to rescue an impairment in protein trafficking. For example, the yeast mutant cell line ypt1^{ts} can be used to identify compounds that rescue cells from the lethal phenotype of a mutant YPT1 allele (see, e.g., Examples and Schmitt et al. (1988) Cell 53:635-47). The activity may be measured, for example, in a whole yeast cell assay using 384-well screening protocol and an optical density measurement.

Table 1 details human orthologs of the yeast genes YPT1 and SAR1. As detailed herein, a cell (e.g., a mammalian cell or a yeast cell) that exhibits reduced expression or activity of a protein required for protein trafficking (e.g., a protein of Table 1) can be used to screen candidate agents for their ability to rescue the cell from a protein trafficking defect.

TABLE 1
Human Counterparts of Yeast Genes YPT1 and SAR1

| | | DNA Accession | Protein Accession |
|---|---|---|---|
| Yeast Gene | Human Gene | Number | Number |
| Name | Name | (Human Gene) | (Human Gene) |
| YPT1 | Rab1a | NM_004161 | NP_004152.1 |
| | Rab1b | NM_030981 | NP_112243.1 |
| | Rab8b | NM_016530 | NP_057614.1 |
| | Rab8a | NM_005370 | NP_005361.2 |
| | Rab10 | NM_016131 | NP_057215.2 |
| | Rab13 | NM_002870 | NP_002861.1 |
| | Rab35 | NM_006861 | NP_006852.1 |
| | Rab11b | NM_004218 | NP_004209.1 |
| | Rab30 | NM_014488 | NP_055303.2 |
| | Rab11a | NM_004663 | NP_004654.1 |
| | Rab3a | NM_002866 | NP_002857.1 |
| | Rab3c | NM_138453 | NP_612462.1 |
| | Rab3d | NM_004283 | NP_004274.1 |
| | Rab3b | NM_002867 | NP_002858.2 |
| | Rab2 | NM_002865 | NP_002856.1 |
| | Rab43 | NM_198490 | NP_940892.1 |
| | Rab4a | NM_004578 | NP_004569.2 |
| | Rab2b | NM_032846 | NP_116235.2 |
| | Rab4b | NM_016154 | NP_057238.2 |
| | Rab25 | NM_020387 | NP_065120.1 |
| | Rab14 | NM_016322 | NP_057406.2 |
| | Rab37 | NM_001006638 | NP_001006639.1 |
| | Rab18 | NM_021252 | NP_067075.1 |
| | Rab5b | NM_002868 | NP_002859.1 |
| | Rab33a | NM_004794 | NP_004785.1 |
| | Rab26 | NM_014353 | NP_055168.2 |
| | Rab5a | NM_004162 | NP_004153.2 |
| | Rab19b | NM_001008749 | NP_001008749.1 |
| | Rab5c | NM_201434 | NP_958842.1 |
| | Rab33b | NM_031296 | NP_112586.1 |
| | Rab39b | NM_171998 | NP_741995.1 |
| | Rab39 | NM_017516 | NP_059986.1 |
| | Rab31 | NM_006868 | NP_006859.2 |
| | Rab15 | NM_198686 | NP_941959.1 |
| | Rab40c | NM_021168 | NP_066991.2 |
| | Rab27b | NM_004163 | NP_004154.2 |
| | Rab22a | NM_020673 | NP_065724.1 |
| | Rab6b | NM_016577 | NP_057661.2 |
| | Rab40b | NM_006822 | NP_006813.1 |
| | Rasef | NM_152573 | NP_689786.2 |
| | Rab21 | NM_014999 | NP_055814.1 |
| | Rab27a | NM_183236 | NP_899059.1 |
| | Loc286526 | NM_001031834 | NP_001027004.1 |
| | Rab40a | NM_080879 | NP_543155.2 |
| | Rab6a | NM_198896 | NP_942599.1 |
| | Rab17 | NM_022449 | NP_071894.1 |
| | Rab6c | NM_032144 | NP_115520.1 |
| | Rab7 | NM_004637 | NP_004628.4 |
| | Rab9a | NM_004251 | NP_004242.1 |
| | Rab7l1 | NM_003929 | NP_003920.1 |
| | Rab9b | NM_016370 | NP_057454.1 |
| | Rab34 | NM_031934 | NP_114140.2 |
| | Rab7b | NM_177403 | NP_796377.2 |
| | Rab41 | NM_001032726 | NP_001027898.1 |
| | Rab23 | NM_183227 | NP_899050.1 |
| | Rab32 | NM_006834 | NP_006825.1 |
| | Rab38 | NM_022337 | NP_071732 |
| | Rab36 | NM_004914 | NP_004905 |
| | Rab28 | NM_001017979 | NP_001017979 |
| | Rab20 | NM_017817 | NP_060287 |
| | Rab12 | NM_001025300 | NP_001020471 |
| SAR1 | Sar1a | NM_020150 | NP_064535 |
| | Sar1b | NM_001033503 | NP_001028675 |

The activity of compounds described herein as modulators of alpha-synuclein toxicity can be measured in standard assays (see, e.g., U.S. patent application Ser. No. 10/826,157, filed Apr. 16, 2004; U.S. Patent Application Publication No. 2003/0073610). The activity can be measured in a whole yeast cell assay using 384-well screening protocol and an optical density measurement. Expression of human alpha-synuclein in yeast inhibits growth in a copy-number dependent manner (see, e.g., Outeiro, et al. (2003) Science 302(5651):1772-5). Expression of one copy of alpha-synuclein:GFP has no effect on growth, while two copies result in complete inhibition. The cessation of growth is accompanied by a change in alpha-synuclein:GFP localization. In cells with one copy, alpha-synuclein:GFP associates with the plasma membrane in a highly selective manner. When expression is doubled, alpha-synuclein migrates to the cytoplasm where it forms large inclusions that are similar to Lewy bodies seen in diseased neurons.

In addition, efficacy of a compound can be evaluated before (first in time), concomitantly or subsequently to the above-mentioned test modalities by monitoring, e.g., (i) modulation (e.g., an improvement) of the stability of a trafficking defective protein, (ii) modulation (e.g., an improvement) of proper, physiological trafficking of the trafficking defective protein, or (iii) modulation (e.g., a restoration) in one or more functions of a trafficking defective protein. For example, in some cases, proteins (e.g., protein mutants such as ΔF508 CFTR) are prematurely degraded. Thus, the efficacy of a given compound to modulate protein trafficking can be determined by monitoring the stability of a protein in the presence as compared to the absence of the compound. For example, cells expressing a trafficking defective protein (e.g., expressing endogenously or expressing an exogenous transgene encoding a trafficking defective protein such as ΔF508 CFTR) can be cultured in the presence or absence of a compound for at least 1 hour (e.g., at least 2 hours, at least 4 hours, at least 6 hours, at least 8 hours, at least 12 hours, at least 16 hours, at least 24 hours, at least 36 hours, or at least 48 hours). Cell lysates can be prepared from the different populations of cells, suspended in Laemmli buffer (with or without reducing agent) and subjected to sodium dodecyl sulfate-polyacrylamide gel electrophoresis (SDS-PAGE). Using antibodies that specifically recognize the trafficking defective protein (e.g., CFTR), the amount of the protein in the presence as compared to in the absence of a compound can be determined by western or dot-blotting techniques. An increase in the amount of a trafficking defective protein in the presence of a compound as compared to in the absence of the compound indicates that the compound modulates (e.g., stabilizes) a trafficking defective protein (Vij et al. (2006) J. Biol. Chem. 281(25):17369-17378). Where a modified state (e.g., glycosylation or phosphorylation) of a protein is indicative of increased stability, a change in the modified state of a protein can also be used to determine if a compound stabilizes the trafficking defective protein. For example, the amount of glycosylated CFTR (e.g., ΔF508 CFTR) can be assessed in the presence as compared to the absence of a compound. An increase in the glycosylated form of the protein is an indicated that the compound has stabilized CFTR (e.g., ΔF508 CFTR).

It is understood that routine adaptation of this assay can be used to monitor any trafficking defective protein. Furthermore, steady-state levels (e.g., protein turnover or the degradation rate) of a protein can also be monitored in the presence and absence of a compound (e.g., see Van Goor et al. (2006) Am. J. Physiol. Lung. Cell Mol. Physiol. 290:L1117-L1130).

Another method of determining modulation of a trafficking defective protein is an in situ staining method. For example, where a protein (e.g., ΔF508 CFTR or G601S-hERG) is prematurely degraded before reaching the cell surface, the efficacy of a compound to modulate the trafficking defective protein can be determined as a change (e.g., an increase) in the amount of surface expression of the protein. Thus, an increase in the amount protein expression at the cell surface in the presence of a compound as compared to the surface expression in the absence of a compound indicates that compound modulates (e.g., stabilizes) the trafficking defective protein. Immunostaining methods are well known to those of skill in the art and include embodiments where the cells are still viable (e.g., confocal microscopy of live cells such as mammalian cells) or staining of fixed cells (e.g., immunohistochemistry). The cells can be attached to a solid support (e.g., a tissue culture plate or poly-lysine coated glass slide) or can be in solution (e.g., for fluorescence assisted cell sorting (FACS) analysis). A primary antibody specific for a trafficking defective protein are applied (e.g., administered, delivered, contacted) to cells. The primary antibody itself can be labeled with a detectable label (e.g., a different colored fluorophore (e.g., rhodamine, texas red, FITC, Green fluorescent protein, Cy3, Cy5). Alternatively, a secondary agent, such as a secondary antibody, can be detectably labeled and the primary antibody unlabeled. The primary antibody can also be conjugated to a first member of a binding pair (e.g., biotin or streptavidin) and the second member of the binding pair detectably labeled. Use of an appropriate microscope (e.g., a confocal microscope) with the appropriate optical filters can identify the position of the labeled antibodies in a given cell. An increase in signal from the detectable label from the cell surface indicates that more protein is expressed on the cell surface. Of course, it is understood that this method can be applied to trafficking defective proteins that localize to other compartments (e.g., organelles such as nucleus, lysosome, ER, Golgi, or mitochondria) of the cell. It can be useful to use another antibody or dye to identify another control protein known to localize to the given compartment of interest. Typically, the second protein is labeled with a different detectable label than the trafficking defective protein of interest. The position of both labels is then determined by the preceding methods. When each of the positions of the two proteins are determined (i.e., the location of their respective detectable label within the cell as determined by antibody binding), if they are found to occupy the same space, the two proteins are said to co-localize and thus, the trafficking defective protein has localized to the proper cellular position (i.e., when two proteins co-localize in the absence of a compound but do not co-localize in the presence of a compound, this can indicate that the compound has inhibited the interaction between the two proteins). Examples of this method are described in, for example, Morello et al. (2000) J. Clin. Invest. 105(7):887-895 and Liu et al. (2003) Proc. Natl. Acad. Sci. USA 100(26):15824-15829. Optionally the cells can be fixed, for example, using paraformaldehyde or formaldehyde, and permeabilized using a detergent (e.g., Triton-X100).

The efficacy of a compound to modulate a trafficking defective protein can also be assessed by monitoring an increase in the activity of the trafficking defective protein. For example, the ΔF508 CFTR is a PKA-regulated chloride channel, and thus an increase in the stability of the CFTR protein can be determined by an increase in, e.g., membrane potential response to forskolin or induction of cAMP-mediated chloride efflux (see, e.g., Vij et al. (2006) J. Biol. Chem. 281(25):17369-17378 and Van Goor et al. (2006) Am. J. Physiol. Lung. Cell Mol. Physiol. 290:L1117-L1130). Alpha-galactosidase-A, the trafficking defective protein in Fabry's disease, is an enzyme that metabolizes certain lipids. Therefore, the efficacy of a compound to modulate alpha-galactosidase-A can be determined by assessing the cellular activity of alpha-galactosidase in the presence as compared to in the absence of a compound. An increase in activity in the presence of the compound as compared to in the absence of the compound indicates that compound modulates (e.g., stabilizes) the alpha-galactosidase-A protein. Methods of monitoring for alpha-galactosidase activities in cells can be found in, e.g., Ioannou et al. (1998) Biochem. J. 332:789-797. Methods for monitoring the in vitro and in vivo enzymatic activities of trafficking defective proteins causative of their respective disorder characterized by impaired protein traffickings, other than CFTR and alpha-galactosidase-A, are known in the art.

Protein trafficking (e.g., endoplasmic reticulum-mediated protein trafficking) can also be detected and measured using in vitro (cell-free) methods. Thus, the efficacy of a compound to modulate, e.g., a trafficking defective protein or various steps of protein trafficking (e.g., formation or docking of COPII vesicles) can be determined using such in vitro methods. Suitable in vitro methods for detecting or measuring endoplasmic-reticulum mediated protein trafficking are described in, e.g., Rexach et al. (1991) J Cell Biol. 114(2):219-229; Segev (1991) Science 252(5012):1553-1556; Balch et al. (1984) Cell 39(2 Pt 1):405-416; Wattenberg (1991) J Electron Microsc Tech 17(2):150-164; Beckers et al. (1989) J. Cell Biol. 108(4):1245-1256; and Moreau et al. (1991) J Biol. Chem 266(7):4322-4328, the contents of each of which are incorporated herein by reference in their entirety. For example, transfer of a protein of interest from endoplasmic reticulum to Golgi can be detected or measured. First, a reporter protein is labeled in a cell, e.g., by metabolically labeling the protein using ³⁵S-methionine or by expressing a detectably-labeled form of the protein in a cell (a fusion protein comprising the protein of interest and green fluorescent protein). “Donor” membrane fractions containing endoplasmic reticulum can be obtained from the cells containing labeled protein. “Acceptor” membrane fractions containing Golgi apparatus can be prepared from cells not containing labeled protein. Transport of the labeled protein is accompanied by post-translational modification. Often the reporter protein is a glycoprotein whose carbohydrate chains are modified during ER to Golgi transport. Acceptor and donor fractions are mixed and incubated with required cofactors. Transport is monitored by the increase in the post-tranlationally modified form of the labeled protein. Methods for detecting the post-translationally modified labeled protein are described herein and can include western,dot blotting, lectin binding, and suspectability to glycosidases. When the detectable label is a fluorescent or luminescent label, a fluorimeter or luminometer can be utilized. When the detectable label is a radioactive label (see below), scintillation counter, X-ray film, or radiometer. It is understood that a protein need not be detectably labeled. A protein initially present in the Donor fraction (e.g., a protein specifically expressed in the Donor cell population), but not present in the Acceptor fraction can be distinguished using, e.g., western blotting techniques.

In vitro methods of detecting protein trafficking (e.g., endoplasmic reticulum-mediated protein trafficking) can also involve measuring vesicle budding, uncoating, tethering, or docking or fusion with the Golgi apparatus (see, e.g., Rexach et al., supra, and Bonifacino et al. (2004) Cell 116:153-166).

To determine if a compound modulates the in vitro transfer of a protein from endoplasmic reticulum to Golgi (e.g., any step of the transfer of a protein from endoplasmic reticulum to Golgi), a compound can be contacted to the Acceptor fraction, Donor fraction, or both before or during the incubation. The compound could be added to either Donor or Acceptor cell populations prior to preparing the membrane fractions. As described herein (see, e.g., Examples), compounds that inhibit the proteasome (e.g., proteosome expression or activity) can also be screened through the assays described herein (e.g., ypt1ts mutant assay) to determine if they rescue endoplasmic reticulum-mediated transport. In vitro and in vivo (cell-based) methods of detecting and/or measuring proteasome activity are known in the art and are described, for example, in Chuhan et al. (2006) Br. J. Cancer 95(8):961-965; Rubin et al. (1998) EMBO J. 17(17):4909-4919; Glickman et al. (1999) Mol. Biol. Rep. 26(1-2):21-8; and Grimes et al. (2005) Int. J. Oncol. 27(4):1047-1052. In vitro methods of determining whether a candidate compound inhibits the proteasome, e.g., proteasome activity, can include contacting isolated proteasome complexes with a candidate compound and measuring the activity of the isolated proteasomes contacted with the candidate compound. A decrease in the activity of a proteasome contacted with a compound as compared to proteasome activity in the absence of the compound indicates that the candidate compound inhibits proteasome activity in vitro. In vivo methods of determining whether a candidate compound inhibits the proteasome can include, e.g., contacting a cell with a candidate compound and measuring the activity of proteasomes in the cell. For example, measuring the turnover of proteins known to be degraded by the proteasome. A decrease in the activity of proteasomes in a cell contacted with a compound as compared to proteasome activity in a cell in the absence of the compound indicates that the candidate compound inhibits proteasome activity in vivo. Examples of proteosome inhibitors include, e.g., MG132, MG15, LLnL, ALLnL, bortezomib/PS-341/Velcade®, NPI-0052, epoxomicin, and lactacystin (Myung et al. (2001) Med. Res. Reviews 21(4):245-273; Montagut et al. (2006) Clin Transl Oncol. 8(5):313-317; and Chuhan et al. (2006) Br. J. Cancer 95(8):961-965).

### F. Methods of Treating a Disorder Characterized by Impaired Protein Trafficking

Disclosed herein are methods to treat or prevent disorders characterized by impaired protein trafficking. In practicing the methods, effective amounts of the compounds or compositions provided herein are administered. Such amounts are sufficient to achieve a therapeutically effective concentration of the compound or active component of the composition in vivo.

In some embodiments, the compounds or compositions described herein are used in methods to inhibit or prevent alpha-synuclein toxicity and/or fibril formation, methods to inhibit or prevent alpha-synuclein fibril growth, and methods to cause disassembly, disruption, and/or disaggregation of alpha-synuclein fibrils and alpha-synuclein-associated protein deposits. The methods can be in vitro or in vivo methods.

In certain embodiments, synucleinopathies treated or whose symptoms are ameliorated by the compounds and compositions described herein include, but are not limited to diseases associated with the formation, deposition, accumulation, or persistence of synuclein fibrils, including alpha-synuclein fibrils. In certain embodiments, such diseases include Parkinson's disease, familial Parkinson's disease, Lewy body disease, the Lewy body variant of Alzheimer's disease, dementia with Lewy bodies, multiple system atrophy, and the Parkinsonism-dementia complex of Guam.

GTP-bound Rab proteins such as Rabl, the homolog of yeast ypt1, are involved in the global regulation of vesicle transport and fusion from the ER to the Golgi. As detailed throughout the specification and in the Examples, compounds identified in the ypt^{ts} mutant rescue screening assay can be useful to stabilize trafficking defective proteins, e.g., by modulating the Rab-ypt1 pathway. Thus, the compounds disclosed herein (and pharmaceutical compositions comprising same) can be useful in methods to treat one or more symptoms of a variety of disorders characterized by impaired protein trafficking.

Types of disorders characterized by impaired protein trafficking that could be treated through the administration of one or more compounds (or pharmaceutical compositions of the same) described herein can include, e.g., hereditary emphysema, α-1-antitrypsin deficiency, hereditary hemochromatosis, oculocutaneous albinism, protein C deficiency, type I hereditary angioedema, congenital sucrase-isomaltase deficiency, Crigler-Najjar type II, Laron syndrome, hereditary Myeloperoxidase, primary hypothyroidism, congenital long QT syndrome, tyroxine binding globulin deficiency, familial hypercholesterolemia, familial chylomicronemia, abeta-lipoproteinema, low plasma lipoprotein a levels, hereditary emphysema with liver injury, congenital hypothyroidism, osteogenesis imperfecta, hereditary hypofibrinogenemia, alpha-1antichymotrypsin deficiency, nephrogenic diabetes insipidus, neurohypophyseal diabetes, insipidus, Charcot-Marie-Tooth syndrome, Pelizaeus Merzbacher disease, von Willebrand disease type IIA, combined factors V and VIII deficiency, spondylo-epiphyseal dysplasia tarda, choroideremia, I cell disease, Batten disease, ataxia telangiectasias, acute lymphoblastic leukemia, acute myeloid leukemia, myeloid leukemia, ADPKD-autosomal dominant polycystic kidney disease, microvillus inclusion disease, tuberous sclerosis, oculocerebro-renal syndrome of Lowe, amyotrophic lateral sclerosis, myelodysplastic syndrome, Bare lymphocyte syndrome, Tangier disease, familial intrahepatic cholestasis, X-linked adreno-leukodystrophy, Scott syndrome, Hermansky-Pudlak syndrome types 1 and 2, Zellweger syndrome, rhizomelic chondrodysplasia puncta, autosomal recessive primary hyperoxaluria, Mohr Tranebjaerg syndrome, spinal and bullar muscular atrophy, primary ciliary diskenesia (Kartagener's syndrome), Miller Dieker syndrome, lissencephaly, motor neuron disease, Usher's syndrome, Wiskott-Aldrich syndrome, Optiz syndrome, Huntington's disease, hereditary pancreatitis, anti-phospholipid syndrome, overlap connective tissue disease, Sjogren's syndrome, stiff-man syndrome, Brugada syndrome, congenital nephritic syndrome of the Finnish type, Dubin-Johnson syndrome, X-linked hypophosphosphatemia, Pendred syndrome, persistent hyperinsulinemic hypoglycemia of infancy, hereditary spherocytosis, aceruloplasminemia, infantile neuronal ceroid lipofuscinosis, pseudoachondroplasia and multiple epiphyseal, Stargardt-like macular dystrophy, X-linked Charcot-Marie-Tooth disease, autosomal dominant retinitis pigmentosa, Wolcott-Rallison syndrome, Cushing's disease, limb-girdle muscular dystrophy, mucoploy-saccharidosis type IV, hereditary familial amyloidosis of Finish, Anderson disease, sarcoma, chronic myelomonocytic leukemia, cardiomyopathy, faciogenital dysplasia, Torsion disease, Huntington and spinocerebellar ataxias, hereditary hyperhomosyteinemia, polyneuropathy, lower motor neuron disease, pigmented retinitis, seronegative polyarthritis, interstitial pulmonary fibrosis, Raynaud's phenomenon, Wegner's granulomatosis, preoteinuria, CDG-Ia, CDG-Ib, CDG-Ic, CDG-Id, CDG-Ie, CDG-If, CDG-IIa, CDG-IIb, CDG-IIc, CDG-IId, Ehlers-Danlos syndrome, multiple exostoses, Griscelli syndrome (type 1 or type 2), or X-linked non-specific mental retardation. In addition, disorders characterized by impaired protein trafficking can also include lysosomal storage disorders such as, but not limited to, Fabry disease, Farber disease, Gaucher disease, GM₁-gangliosidosis, Tay-Sachs disease, Sandhoff disease, GM₂ activator disease, Krabbe disease, metachromatic leukodystrophy, Niemann-Pick disease (types A, B, and C), Hurler disease, Scheie disease, Hunter disease, Sanfilippo disease, Morquio disease, Maroteaux-Lamy disease, hyaluronidase deficiency, aspartylglucosaminuria, fucosidosis, mannosidosis, Schindler disease, sialidosis type 1, Pompe disease, Pycnodysostosis, ceroid lipofuscinosis, cholesterol ester storage disease, Wolman disease, Multiple sulfatase, galactosialidosis, mucolipidosis (types II ,III, and IV), cystinosis, sialic acid storage disorder, chylomicron retention disease with Marinesco-Sjogren syndrome, Hermansky-Pudlak syndrome, Chediak-Higashi syndrome, Danon disease, or Geleophysic dysplasia.

Symptoms of disorder characterized by impaired protein trafficking are numerous and diverse and can include one or more of, e.g., anemia, fatigue, bruising easily, low blood platelets, liver enlargement, spleen enlargement, skeletal weakening, lung impairment, infections (e.g., chest infections or pneumonias), kidney impairment, progressive brain damage, seizures, extra thick meconium, coughing, wheezing, excess saliva or mucous production, shortness of breath, abdominal pain, occluded bowel or gut, fertility problems, polyps in the nose, clubbing of the finger/toe nails and skin, pain in the hands or feet, angiokeratoma, decreased perspiration, corneal and lenticular opacities, cataracts, mitral valve prolapse and/or regurgitation, cardiomegaly, temperature intolerance, difficulty walking, difficulty swallowing, progressive vision loss, progressive hearing loss, hypotonia, macroglossia, areflexia, lower back pain, sleap apnea, orthopnea, somnolence, lordosis, or scoliosis. It is understood that due to the diverse nature of the trafficking defective proteins and the resulting disease phenotypes (e.g., a disorder characterized by impaired protein trafficking), a given disorders will generally present only symptoms characteristic to that particular disorder. For example, a patient with cystic fibrosis can present a particular subset of the above-mentioned symptoms such as, but not limited to, persistent coughing, excess saliva and mucus production, wheezing, coughing, shortness of breath, enlarged liver and/or spleen, polyps of the nose, diabetes, fertility problems, increased infections (e.g., respiratory infections such as pneumonias), or occluded gut or bowel.

Depending on the specific nature of the disorder, a patient can present these symptoms at any age. In many cases, symptoms can present in childhood or in early adulthood. For example, symptoms of cystic fibrosis often present at birth when a baby's gut becomes blocked by extra-thick muconium.

Following administration of one or more of the disclosed compounds (or pharmaceutical compositions) to a subject (e.g., a human patient), the efficacy of the treatment in ameliorating one or more symptoms of a disorder characterized by impaired protein trafficking can be assessed by comparing the number and/or severity of one or more symptoms presented by a patient before and after treatment. Alternatively, where administration of the compounds is used to prevent the occurrence of a disorder characterized by impaired protein trafficking, treatment efficacy can be assessed as a delay in presentation of, or a failure to present, one or more symptoms of a disorder characterized by impaired protein trafficking. The efficacy of a treatment (e.g., a compound or composition described herein) over time (e.g., a progressive improvement) in ameliorating one or more symptoms of a disorder characterized by impaired protein trafficking can be determined by assessing, e.g., the number or severity of one or more symptoms at multiple time points following treatment. For example, a subject (e.g., a patient) can have an initial assessment of the severity of his or her disorder (e.g., the number or severity of one or more symptoms of a disorder characterized by impaired protein trafficking), administered treatment, and then assessed subsequently to the treatment two or more times (e.g., at one week and one month; at one month at two months; at two weeks, one month, and six months; or six weeks, six months, and a year). Where one or more compounds or compositions are administered to a subject for a limited period of time (e.g., a predetermined duration) or number of administrations, the effect of treatment on ameliorating one or more symptoms of a disorder characterized by impaired protein trafficking can be assessed at various time points after the final treatment. For example, following the last administration of a dose of one or more compounds, the number or severity of a patient's symptoms can be assessed at 1 month (e.g., at 2 months, at 6 months, at one year, at two years, at 5 years or more) subsequent to the final treatment.

The efficacy of a treatment with one or more compounds (or compositions) described herein on one or more symptoms of a disorder characterized by impaired protein trafficking can be assessed as a monotherapy or as part of a multi-therapeutic regimen. For example, the compound(s) can be administered in conjunction with other clinically relevant treatments for disorder characterized by impaired protein traffickings including, but not limited to, physical or respiratory therapy, antibiotics, anti-asthma therapies, cortisteroids, vitamin supplements, pulmozyme treatments, Cerezyme®, Ceredase®, Myozyme®, insulin, Fabryzyme®, dialysis, transplants (e.g., liver or kidney), stool softeners or laxatives, anti-blot clotting agents (anti-coagulants), pain medications, and/or angioplasty. It is understood that due to the diverse activities of trafficking defective proteins and the diverse clinical manifestations of the associated disorders (e.g., Fabry's disease, cystic fibrosis, Gaucher's disease, Pompe disease, and the like) the “other clinically relevant treatments” can also include those beyond those above. For example, other or additional clinically relevant treatments for cystic fibrosis include, e.g., antibiotics, pulmozyme treatments, vitamin supplements, stool softeners or laxatives, insulin for cystic-fibrosis related diabetes, anti-asthma therapies, or corticosteroids.

A compound or pharmaceutical composition thereof described herein can be administered to a subject as a combination therapy with another treatment (another active ingredients), e.g., a treatment for a disorder characterized by impaired protein trafficking such as cystic fibrosis or a lysosomal storage disease. For example, the combination therapy can include administering to the subject (e.g., a human patient) one or more additional agents that provide a therapeutic benefit to the subject who has, or is at risk of developing, (or suspected of having) a disorder characterized by impaired protein trafficking such as cystic fibrosis. Thus, the compound or pharmaceutical composition and the one or more additional agents are administered at the same time. Alternatively, the compound can be administered first in time and the one or more additional agents administered second in time. The one or more additional agents can be administered first in time and the compound administered second in time. The compound can replace or augment a previously or currently administered therapy (also, see below). For example, upon treating with a compound of the invention, administration of the one or more additional agents can cease or diminish, e.g., be administered at lower levels. Administration of the previous therapy can also be maintained. In some instances, a previous therapy can be maintained until the level of the compound (e.g., the dosage or schedule) reaches a level sufficient to provide a therapeutic effect. The two therapies can be administered in combination.

The compounds and compositions described herein may also be administered in combination, or sequentially, with another therapeutic agent known for treatment or amelioration of one or more symptoms of diseases or disorders characterized by impaired protein trafficking. For example, when the compounds described herein are administered to treat a synucleinopathy, they can optionally be administered with a therapeutic agent such as donepezil hydrochloride (Aracept), rivastigmine tartrate (Exelon), tacrine hydrochloride (Cognex), and/or galantamine hydrobromide (Reminyl).

It will be appreciated that in instances where a previous therapy is particularly toxic (e.g., a treatment for disorder characterized by impaired protein trafficking carrying significant side-effect profiles) or poorly tolerated by a subject (e.g., a patient), administration of the compound can be used to offset and/or lessen the amount of the previous therapy to a level sufficient to give the same or improved therapeutic benefit, but without the toxicity.

In some instances, when the subject is administered a compound or pharmaceutical composition of the invention, the first therapy is halted. The subject can be monitored for a first pre-selected result, e.g., an improvement in one or more symptoms of a disorder characterized by impaired protein trafficking such as any of those described herein (e.g., see above). In some cases, where the first pre-selected result is observed, treatment with the compound is decreased or halted. The subject can then be monitored for a second pre-selected result after treatment with the compound is halted, e.g., a worsening of a symptom of disorder characterized by impaired protein trafficking. When the second pre-selected result is observed, administration of the compound to the subject can be reinstated or increased, or administration of the first therapy is reinstated, or the subject is administered both a compound and first therapy, or an increased amount of the compound and the first therapeutic regimen.

Methods of assessing the effect of a therapy (e.g., a compound or composition of the invention) are known in the art of medicine and include assessing the change (e.g., the improvement) in one or more symptoms of a disorder characterized by impaired protein trafficking such as any of those described herein (see above). In addition, while the invention is not limited by any particular theory or mechanism of action, because the compounds identified herein can function at the molecular level to correct the disorder characterized by impaired protein trafficking, assessing the effect of a therapy on patient having a disorder characterized by impaired protein trafficking can be done by assessing, e.g., (i) an improvement of the stability of a trafficking defective protein, (ii) improvement of proper, physiological trafficking of the trafficking defective protein, or (iii) a restoration in one or more functions of a trafficking defective protein (see above under “E. Evaluation of the Activity of the Compounds”).

In particular, efficacy of treatment (e.g., administration of one or more compounds or pharmaceutical compositions described herein) of cystic fibrosis can be monitored, e.g., by performing a “sweat test” before an after treatment. The sweat test is generally conducted by a physician or medical practitioner. A colorless, odorless chemical is placed on the skin, which causes it to sweat, and a device collects the sweat. A sweat test can take 30 minutes to 1 hour, depending on how long it takes to collect the subject's perspiration. Chloride levels in the subject's perspiration are measured (e.g., using a Sweat-Chek™ Sweat Conductivity Analyzer, Discovery Diagnostics, Ontario, Canada) and, for example, a relative score of <40 indicates normality, a score of 40-59 is an intermediate range, and a score of >60 indicates that the subject still has profound disease. Efficacy of a treatment of cystic fibrosis can also be determined using a nasal potential difference (NPD) test. The test is especially useful for subjects (e.g., patients) who have normal chloride levels as determined by sweat tests. The NPD test requires 2 electrodes, connected to a voltmeter such as the Tholy-Medicap® device), one placed on the nasal mucosa of the inferior turbinate and the other placed subcutaneously on the forearm. Generally, a reading less than −40 mV is considered abnormal. Thus, a patient who's NPD test readings improve to over −40 mV can be one considered to improve (see, for example, Domingo-Ribas et al. (2006) Arch Bronconeumol. 42:33-38).

### G. Methods of Producing a Protein

The compounds described herein enhance endoplasmic reticulum-mediated transport and thus can be used in methods to enhance protein production in a cell. The protein produced by the methods can be a naturally occurring or a non-naturally occurring protein. The protein can be produced naturally by a cell (e.g., without any genetic manipulation of the cell), can be encoded by a heterologous nucleic acid introduced into a cell, or can be produced by a cell following the insertion or activation of sequences that regulate expression of a gene encoding the protein.

A “heterologous nucleic acid” refers to a nucleotide sequence that has been introduced into a cell by the use of recombinant techniques. Accordingly, a “heterologous nucleic acid” present in a given cell does not naturally occur in the cell (e.g., has no corresponding identical sequence in the genome of the cell) and/or is present in the cell at a location different than that where a corresponding identical sequence naturally exists (e.g., the nucleotide sequence is present in a different location in the genome of the cell or is present in the cell as a construct not integrated in the genome).

Any protein that is produced by a cell can be used in the methods described herein. For example, proteins such as cytokines, lymphokines, and/or growth factors can be produced. Examples of such proteins include, but are not limited to, Erythropoietin, Interleukin 1-Alpha, Interleukin 1-Beta, Interleukin-2, Interleukin-3, Interleukin-4, Interleukin-5, Interleukin-6, Interleukin-7, Interleukin-8, Interleukin-9, Interleukin-10, Interleukin-11, Interleukin-12, Interleukin-13, Interleukin-14, Interleukin-15, Lymphotactin, Lymphotoxin Alpha, Monocyte Chemoattractant Protein-1, Monocyte Chemoattractant Protein-2, Monocyte Chemoattractant Protein-3, Megapoietin, Oncostatin M, Steel Factor, Thrombopoietin, Vascular Endothelial Cell Growth Factor, Bone Morphogenetic Proteins, Interleukin-1 Receptor Antagonist, Granulocyte-Colony Stimulating Factor, Leukemia Inhibitory Factor, Granulocyte-Macrophage Colony-Stimulating Factor, Macrophage Colony-Stimulating Factor, Interferon Gamma, Interferon Beta, Fibroblast Growth Factor, Tumor Necrosis Factor Alpha, Tumor Necrosis Factor Beta, Transforming Growth Factor Alpha, Gonadotropin, Nerve Growth Factor, Platelet-Derived Growth Factor, Macrophage Inflammatory Protein 1 Alpha, Macrophage Inflammatory Protein 1 Beta, and Fas Ligand. Cells producing a non-naturally occurring, variant of any the above polypeptides can also be used in the methods described herein.

In addition to the proteins described above, the methods described herein can also be used to produce a fusion protein that contains all or a portion of a given protein fused to a sequence of amino acids that direct secretion of the fusion protein from a cell. In some cases, such fusion proteins can allow for the secretion of a polypeptide sequence that is not typically secreted from a cell. For example, all or a portion of a protein (e.g., a membrane associated protein such as a receptor or an intracellular protein) can be fused to a portion of an immunoglobulin molecule (e.g., to the hinge region and constant region CH2 and CH3 domains of a human IgG1 heavy chain).

The protein produced by the methods described herein can be an antibody or an antigen-binding fragment of an antibody. The antibody can be directed against an antigen, e.g., a protein antigen such as a soluble polypeptide or a cell surface receptor. For example, the antibody can be directed against a cell surface receptor involved in immune cell activation, a disease-associated antigen, or an antigen produced by a pathogen. The term “antibody” refers to an immunoglobulin molecule or an antigen-binding portion thereof As used herein, the term “antibody” refers to a protein containing at least one, for example two, heavy chain variable regions (“VH”), and at least one, for example two, light chain variable regions (“VL”). The VH and VL regions can be further subdivided into regions of hypervariability, termed “complementarity determining regions” (“CDR”), interspersed with regions that are more conserved, termed “framework regions” (FR). The antibody can further include a heavy and light chain constant region, to thereby form a heavy and light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region contains three domains, CH1, CH2, and CH3. The light chain constant region contains one domain, CL. The variable region of the heavy and light chains contains a binding domain that interacts with an antigen.

The protein can be a fully human antibody (e.g., an antibody made in a mouse genetically engineered to produce an antibody from a human immunoglobulin sequence), a humanized antibody, or a non-human antibody, e.g., a rodent (mouse or rat), goat, or primate (e.g., monkey) antibody.

The following are examples of the practice of the invention. They are not to be construed as limiting the scope of the invention in any way.

### Examples

Compounds that Restore Growth of a ypt1^{ts} Mutant

The yeast mutant cell line ypt1^{ts} suppresses, in a temperature dependent fashion, the dominant-lethal phenotype of a mutant YPT1 allele (Schmitt et al. (1988) Cell 53:635-47). ypt1^{ts} cells grow normally at temperatures up to 30° C., but are growth arrested at 37° C. (Id.). At the non-permissive temperature of 37° C., ypt1^{ts} mutants accumulate ER membranes, small vesicles, and unprocessed invertase and exhibit cytoskeletal defects and enhanced calcium uptake (Id.). ypt1^{ts} mutant cells can be rescued from growth arrest by the provision of extracellular calcium (Id.).

A library of compounds (“The Spectrum Collection”) obtained from Microsource Discovery Systems, Inc. (Gaylordsville, Conn.) was screened to assess the ability of individual compounds to restore growth of ypt1^{ts} cells. Screening was carried out using ypt1^{ts} cells containing a mutation in the pdr5 gene (resulting in inactivation of a membrane efflux pump). The effect of the compounds was measured in pdr5 ypt1^{ts} cells cultured at 37° C. Compounds that were found to rescue pdr5 ypt1^{ts} toxicity are depicted in Table 2 and Table 3. The numerical values presented in the tables represent the optical density recorded in individual wells following culture of the pdr5 ypt1^{ts} cells with various concentrations (25 μM, 12.5 μM, 6.25 μM, 3.13 μM, 1.56 μM, 0.78 μM, 0.39 μM, and 0 μM in Table 2 and 50 μM, 12.5 μM, 3.13 μM, and 0 μM in Table 3) of a test compound. An increased optical density reading as compared to background (0 μM) indicates that the cells exhibited enhanced viability in the presence of the test compound. The numerical value in the minimal rescue concentration (“MRC”) column of Table 2 indicates the lowest concentration of compound tested that was found to result in growth of pdr5 ypt1^{ts} cells above the background level (i.e., above the level detected with 0 μM of compound). The finding that the compounds depicted in Table 2 and Table 3 can rescue the ypt1^{ts} protein trafficking defect indicates that the compounds can be used to treat or prevent a variety of disorders (including synucleinopathies such as Parkinson's disease) characterized by impaired protein trafficking.

### Other Embodiments

It is to be understood that, while the invention has been described in conjunction with the detailed description thereof, the foregoing description is intended to illustrate and not limit the scope of the invention. Other aspects, advantages, and modifications of the invention are within the scope of the claims set forth below.

TABLE 2
Compounds That Restore Growth of pdr5 ypt1ts Cells

| | | | | ypt1-ts | | | | | | | | |
|---|---|---|---|---|---|---|---|---|---|---|---|---|
| Name | References | Group | Molecular Structure | MRC | 25 | 12.5 | 6.25 | 3.13 | 1.56 | 0.78 | 0.39 | 0 |
| PRISTIMERIN | 1. Celastrols as Inducers of the Heat Shock Response and Cytoprotection. Journal of Biological Chemistry (2004), 279(53), 56053- 56060. 2. The relationship between structure and antimicrobial activity in quinones from the Celastraceae. Biochemical Systernatics and Ecology (1990), 18(1), 25-8. | 6 | 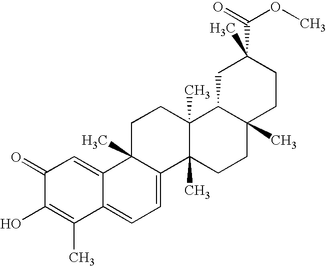 | 0.39 | 0.677 | 0.676 | 0.68 | 0.703 | 0.669 | 0.332 | 0.534 | 0.058 |
| 3beta- ACETOXYDEOXYANG OLENSIC ACID, METHYL ESTER | | 1 | 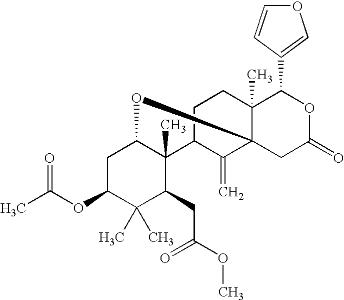 | 1.56 | 0.553 | 0.571 | 0.538 | 0.374 | 0.133 | 0.096 | 0.129 | 0.058 |
| 4′-METHOXYFLAVONE | 1. Benzoflavone activators of the cystic fibrosis transmembrane conductance regulator: towards a pharmacophore model for the nucleotide- binding domain. Bioorganic & Medicinal Chemistry (2003), 11(18), 4113- 4120. 2. Chalcones and flavonoids as anti- Tuberculosis agents. Bioorganic & Medicinal Chemistry (2002), 10(8), 2795-2802. | 4 | 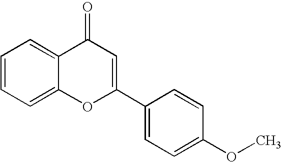 | 1.56 | 0.557 | 0.556 | 0.567 | 0.474 | 0.136 | 0.077 | 0.111 | 0.058 |
| CEDRELONE | 1. Caspase cascade- based methods for identifying therapeutically effective antineoplastic agents, compounds so identified, and pharmaceutical compositions. PCT Int. Appl. (2000), 87 pp. CODEN: PIXXD2 WO 2000045165 | 7 | 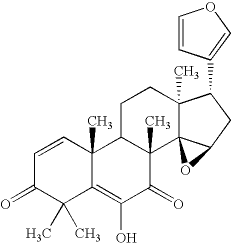 | 1.56 | 0.496 | 0.541 | 0.532 | 0.49 | 0.264 | 0.124 | 0.221 | 0.058 |
| ANGOLENSIC ACID, METHYL ESTER | 1. Inhibition of allergen-induced eosinophil recruitment by natural tetra- nortriterpenoids is mediated by the suppression of IL-5, CCL11/eotaxin and NFkB activation. International Immuno- pharmacology (2005), Volume Date 2006, 6(2), 109-121. 2. Behavioural effects in rodents of methyl angolensate: a triterpenoid isolated from Entandropragma angolense. Pharmacology & Toxicology (Oxford, United Kingdom) (2002), 91(2), 71-76. | 1 | 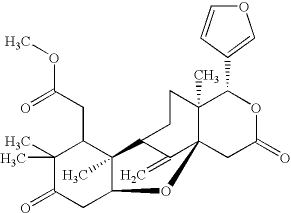 | 3.13 | 0.577 | 0.56 | 0.53 | 0.21 | 0.107 | 0.087 | 0.119 | 0.058 |
| DEOXYANDIROBIN- LACTONE | | 1 | 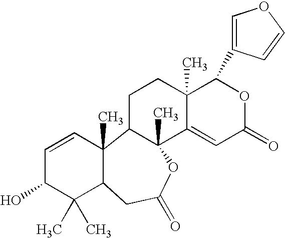 | 3.13 | 0.613 | 0.524 | 0.249 | 0.137 | 0.089 | 0.082 | 0.107 | 0.058 |
| 3-DEOXO-3beta- HYDROXYMEXICANO LIDE 16-ENOL ETHER | | 1 | 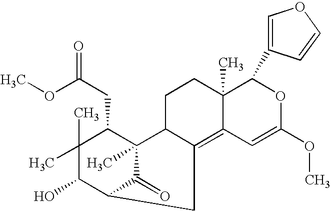 | 3.13 | 0.625 | 0.583 | 0.219 | 0.141 | 0.091 | 0.077 | 0.102 | 0.058 |
| 3alpha-HYDROXY-3- DEOXYANGOLENSIC ACID METHYL ESTER2 | derivative of ANGOLENSIC ACID, METHYL ESTER | 1 | 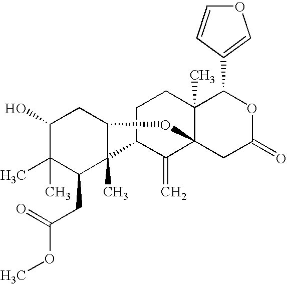 | 3.13 | 0.611 | 0.566 | 0.381 | 0.144 | 0.093 | 0.078 | 0.169 | 0.058 |
| ABIETIC ACID | 1. ABC transporter- based methods for the identification and use of compounds suitable for the treatment of drug- resistant cancer cells. PCT Int. Appl. (2006), 99 pp. CODEN: PIXXD2 WO 2006009765. 2. Comparative enzymology of 11b- hydroxysteroid dehydrogenase type 1 from six species. Journal of Molecular Endocrinology (2005), 35(1), 89-101. | 2 | 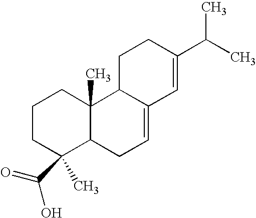 | 3.13 | 0.61 | 0.528 | 0.173 | 0.165 | 0.1 | 0.079 | 0.107 | 0.058 |
| DUARTIN, DIMETHYL ETHER | 1. Inhibitors of prostaglandin biosynthesis from Dalbergia odorifera. Chemical & Pharmaceutical Bulletin (1992), 40(9) 2452-7 | 4 | 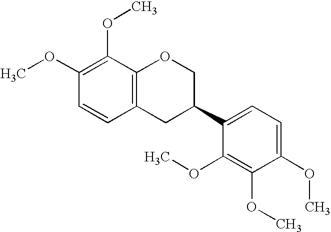 | 3.13 | 0.474 | 0.287 | 0.239 | 0.14 | 0.111 | 0.092 | 0.166 | 0.058 |
| ANTHOTHECOL | Effects of Naturally Occurring Compounds on Fibril Formation and Oxidative Stress of b- Amyloid. Journal of Agricultural and Food Chemistry (2005), 53(22), 8537-8541. | 7 | 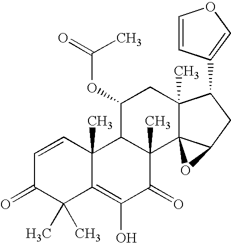 | 3.13 | 0.557 | 0.581 | 0.537 | 0.262 | 0.12 | 0.085 | 0.161 | 0.058 |
| BENZANTHRONE | | 9 | 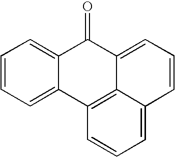 | 3.13 | 0.383 | 0.547 | 0.491 | 0.263 | 0.123 | 0.09 | 0.108 | 0.058 |
| SUXIBUZONE | 1. Prevention and treatment of Alzheimer's disease with Ab42 lowering agents. U.S. Pat. application Publ. (2002), 35 pp., Cont.-in-part of application No. PCTUS/ 01/11956. CODEN: USXXCO US 2002128319 A1 20020912 CAN 137:210976 2. Nonsteroidal antiinflammatory drug (NSAID) and NSAID derivative amyloid Ab42 polypeptide- lowering agents for the treatment of Alzheimer's disease, and screening methods. PCT Int. Appl. (2001), 73 pp. CODEN: PIXXD2 WO 2001078721 A1 20011025 CAN 135:327362 | 15 | 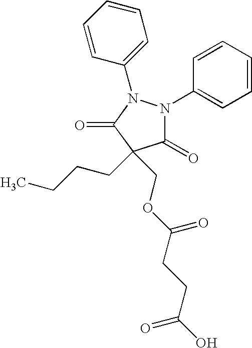 | 3.13 | 0.536 | 0.329 | 0.173 | 0.127 | 0.093 | 0.078 | 0.101 | 0.058 |
| NITRENDIPINE | 1. Pyridine derivatives for inhibiting beta-amyloid production and methods of identifying the compounds. PCT Int. Appl. (2006), 192 pp. CODEN: PIXXD2 WO 2006074419 A2 20060713. 2. Cardiovascular compounds comprising nitric oxide enhancing groups, compositions, and methods of use. PCT Int. Appl. (2006), 140 pp. CODEN: PIXXD2 WO 2006093864 A1 20060908 | 22 | 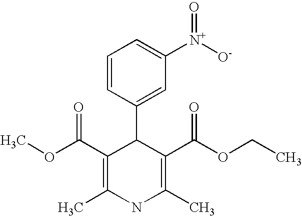 | 3.13 | 0.481 | 0.403 | 0.309 | 0.143 | 0.103 | 0.089 | 0.11 | 0.058 |
| GEMFIBROZIL | 1. PPAR modulators for treatment of CFTR mutation-related diseases. U.S. Pat. application Publ. (2006), 36 pp., Cont.-in-part of application No. PCT/US04/ 013412. CODEN: USXXCO US 2006160867 A1 20060720 2. Preparation of benzisoxazole derivs. as novel LXR ligands useful in treatment of dyslipidemic diseases. U.S. Pat. application Publ. (2006), 58 pp. CODEN: USXXCO US 2006178398 A1 20060810 | | 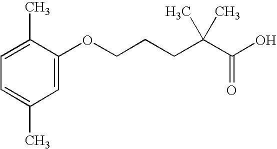 | 3.13 | 0.358 | 0.615 | 0.442 | 0.219 | 0.1 | 0.082 | 0.106 | 0.058 |
| HAEMATOMMIC ACID, ETHYL ESTER | | | 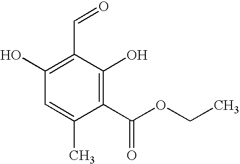 | 3.13 | 0.645 | 0.621 | 0.204 | 0.135 | 0.105 | 0.078 | 0.104 | 0.058 |
| 3,3'- DIINDOLYLMETHANE | | | 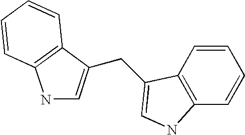 | 3.13 | 0.035 | 0.263 | 0.154 | 0.164 | 0.118 | 0.097 | 0.205 | 0.058 |
| MEXICANOLIDE | 1. Antifungal activity of some B,D- secolimonoids from two meliaceous plants. Journal of Chemical Ecology (1999), 25(4), 923-933. | 1 | 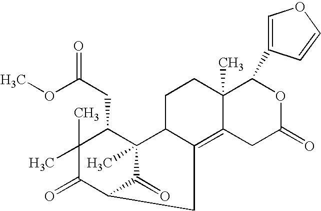 | 6.25 | 0.573 | 0.377 | 0.216 | 0.105 | 0.076 | 0.069 | 0.092 | 0.058 |
| 3alpha-HYDROXY-3- DEOXYANGOLENSIC ACID METHYL ESTER | | 1 | | 6.25 | 0.591 | 0.359 | 0.146 | 0.103 | 0.074 | 0.072 | 0.09 | 0.058 |
| FUSIDIC ACID | 1. Treatment of MRSA infections. Ben-David, Debby; Rubinstein, Ethan. Infectious Diseases Unit, Sheba Medical Center, Tel Hashomer, Israel. Editor(s): Fluit, Ad C.; Schmitz, Franz-Josef. MRSA: Current Perspectives (2003), 275-315. | 3 | 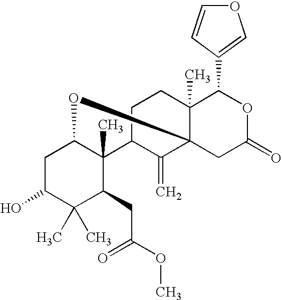 | 6.25 | 0.52 | 0.296 | 0.137 | 0.107 | 0.084 | 0.081 | 0.095 | 0.058 |
| PRISTIMEROL | 1. Celastrols as Inducers of the Heat Shock Response and Cytoprotection. Journal of Biological Chemistry (2004), 279(53), 56053- 56060. 2. Derivatives of pentacyclic nortriterpene quinone methides as compounds useful in the treatment of inflammatory, neuro- degenerative, and neoplastic diseases. U.S. Pat. application Publ. (2004), 4 pp. CODEN: USXXCO US 2004220267 A1 20041104 CAN 141:388762 | 6 | 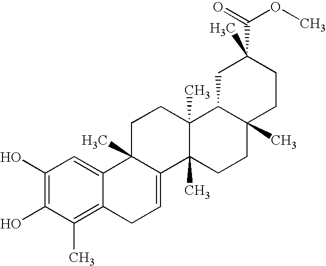 | 6.25 | 0.591 | 0.362 | 0.168 | 0.114 | 0.101 | 0.079 | 0.11 | 0.058 |
| AVOCADENE | 1. Synthesis of all four stereoisomers of antibacterial component of avocado. Agricultural and Biological Chemistry (1982), 46(2), 481-5. | 8 | 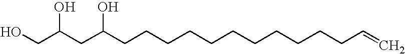 | 6.25 | 0.408 | 0.209 | 0.134 | 0.108 | 0.09 | 0.074 | 0.097 | 0.058 |
| XANTHONE | 1. Synthesis and pharmacological activities of xanthone derivatives as a- glucosidase inhibitors. Bioorganic & Medicinal Chemistry (2006), 14(16), 5683-5690. | 9 | 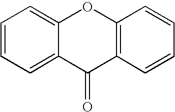 | 6.25 | 0.598 | 0.445 | 0.152 | 0.109 | 0.093 | 0.072 | 0.096 | 0.058 |
| PTERYXIN | 1. Inhibitory effects of coumarin and acetylene constituents from the roots of Angelica furcijuga on D- galactosamine/ lipopol ysaccharide- induced liver injury in mice and on nitric oxide production in lipo- polysaccharide- activated mouse peritoneal macrophages. Bioorganic & Medicinal Chemistry (2006), 14(2), 456-463. | 10 | 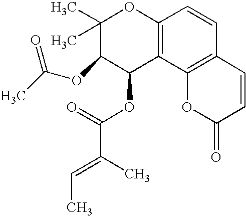 | 6.25 | 0.479 | 0.26 | 0.134 | 0.124 | 0.096 | 0.078 | 0.103 | 0.058 |
| PROPACHLOR | | 13 | 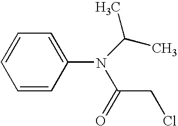 | 6.25 | 0.184 | 0.177 | 0.141 | 0.121 | 0.098 | 0.085 | 0.101 | 0.058 |
| PHENYLBUTAZONE | | 15 | 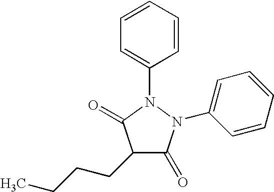 | 6.25 | 0.452 | 0.227 | 0.154 | 0.11 | 0.094 | 0.084 | 0.1 | 0.058 |
| 3,4-DIDESMETHYL-5- DESHYDROXY-3'- ETHOXYSCLEROIN | | 19 | 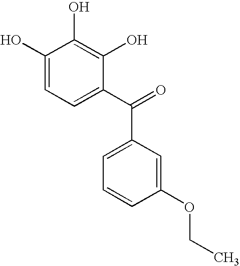 | 6.25 | 0.396 | 0.185 | 0.143 | 0.108 | 0.091 | 0.075 | 0.094 | 0.058 |
| ONONETIN | 1. Peptidomimetic modulators of cell adhesion. U.S. Pat. application Publ. (2004), 280 pp., Cont.-in-part of U.S. Ser. No. 6,982. CODEN: USXXCO US 2004006011 A1 | 20 | 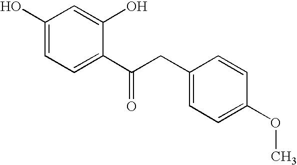 | 6.25 | 0.618 | 0.3 | 0.14 | 0.111 | 0.096 | 0.08 | 0.097 | 0.058 |
| ACETYLCHOLINE | | | 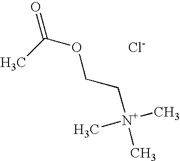 | 6.25 | 0.352 | 0.27 | 0.164 | 0.113 | 0.085 | 0.087 | 0.104 | 0.058 |
| DACTINOMYCIN | | | 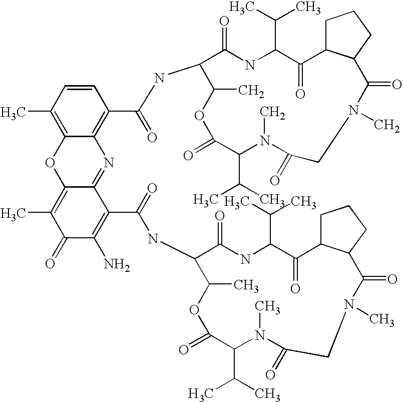 | 6.25 | 0.669 | 0.663 | 0.351 | 0.104 | 0.082 | 0.071 | 0.093 | 0.058 |
| SOLIDAGENONE | 1. Gastroprotective and ulcer-healing effect of new solidagenone derivatives in human cell cultures. Life Sciences (2005), 77(17), 2193-2205. | | 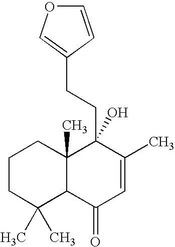 | 6.25 | 0.62 | 0.439 | 0.141 | 0.11 | 0.086 | 0.077 | 0.092 | 0.058 |
| BROMO-3-HYDROXY- 4-(SUCCIN-2-YL)- CARYOLANE gamma- LACTONE | | | 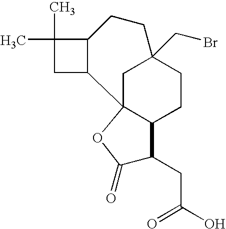 | 6.25 | 0.417 | 0.245 | 0.139 | 0.101 | 0.091 | 0.079 | 0.091 | 0.058 |
| 14-METHOXY-4,4- BISNOR-8,11,13- PODOCARPATRIEN-3- ONE | | 2 | 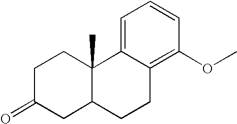 | 12.5 | 0.48 | 0.191 | 0.096 | 0.088 | 0.075 | 0.074 | 0.088 | 0.058 |
| HYDROXY (19)TOTAROL | 1. Antibacterial activity of totarol and its potentiation. Journal of Natural Products (1992), 55(10), 1436-40. | 2 | 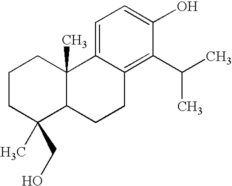 | 12.5 | 0.515 | 0.407 | 0.125 | 0.106 | 0.085 | 0.076 | 0.098 | 0.058 |
| EPIANDROSTANEDIOL | 1. Use of androgens to reduce the likelihood of acquiring or to treat skin aging. PCT Int. Appl. (2006), 49 pp. CODEN: PIXXD2 WO 2006047859 A1 | 3 | 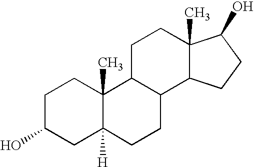 | 12.5 | 0.321 | 0.145 | 0.099 | 0.082 | 0.068 | 0.073 | 0.082 | 0.058 |
| PROGESTERONE | 1. Effects of female steroid hormones on A-type K+ currents in murine colon. Journal of Physiology (Oxford, United Kingdom) (2006), 573(2), 453-468. 2. 17b-Estradiol increases volume, apical surface and elasticity of human endothelium mediated by Na+/H+ exchange. Cardiovascular Research (2006), 69(4), 916-924. | 3 | 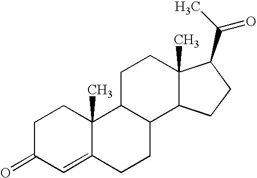 | 12.5 | 0.316 | 0.15 | 0.12 | 0.098 | 0.091 | 0.083 | 0.089 | 0.058 |
| CHLORMADINONE ACETATE | 1. Chlormadinone acetate (CMA) in oral contraception - A new opportunity. European Journal of Contraception & Reproductive Health Care (2005), 10(Suppl. 1), 7-11. | 3 | 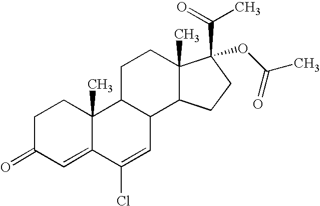 | 12.5 | 0.296 | 0.2 | 0.11 | 0.112 | 0.088 | 0.078 | 0.193 | 0.058 |
| DEOXYSAPPANONE B 7,3'-DIMETHYL ETHER | | 4 | 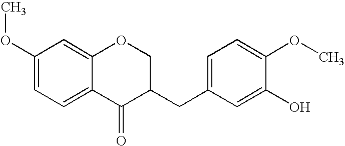 | 12.5 | 0.62 | 0.319 | 0.125 | 0.101 | 0.085 | 0.068 | 0.099 | 0.058 |
| 2-METHOXYXANTHONE | 1. Natural and synthetic xanthones as monoamine oxidase inhibitors: biological assay and 3D-QSAR. Helvetica Chimica Acta (2001), 84(3), 552-570. 2. Xanthanes as inhibitors of growth of human cancer cell lines and Their effects on the proliferation of human lymphocytes In Vitro. Bioorganic & Medicinal Chemistry (2002), 10(12), 3725-3730. | 4 | 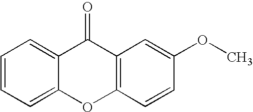 | 12.5 | 0.221 | 0.128 | 0.108 | 0.094 | 0.083 | 0.069 | 0.093 | 0.058 |
| METHOXYVONE | | 4 | 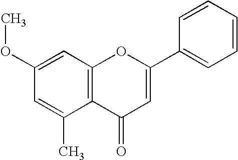 | 12.5 | 0.258 | 0.142 | 0.114 | 0.105 | 0.09 | 0.09 | 0.095 | 0.058 |
| DIETHYLSTILBESTROL | 1. Estrogen inhibition of cystic fibrosis transmembrane conductance regulator mediated chloride secretion. Journal of Pharmacology and Experimental Therapeutics (2000), 295(1), 195-204. | 5 | 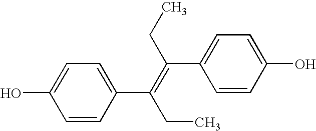 | 12.5 | 0.162 | 0.163 | 0.103 | 0.108 | 0.091 | 0.076 | 0.089 | 0.058 |
| 7-DESACETOXY-6,7- DEHYDROGEDUNIN | | 7 | 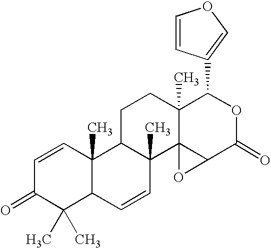 | 12.5 | 0.308 | 0.171 | 0.099 | 0.093 | 0.074 | 0.071 | 0.094 | 0.058 |
| DIHYDROSAMIDIN | 1. Effect of the vasodilating agent dimidin (dihydrosamidin) on the course of cholesterol atherosclerosis in rabbits. Mater. Vses. Konf. Issled. Lek. Rast. Perspekt. Ikh Ispol’z. Proizvod. Lek. Prep. (1972), Meeting Date 1970, 70-6. 2. Treatment of cattle babesiasis with a combination of dimidin and azidin. Izvestiya Sel’- skokhozyaistvenn ykh Nauk (1978), 21(5), 58-61. | 10 | 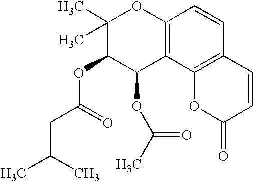 | 12.5 | 0.221 | 0.14 | 0.097 | 0.105 | 0.079 | 0.068 | 0.091 | 0.058 |
| 2',beta- DIHYDROXYCHALCONE | 1. Design of novel bioisosteres of b- diketo acid inhibitors of HIV-1 integrase. Antiviral Chemistry & Chemotherapy (2005), 16(1), 41-61. | 11 | 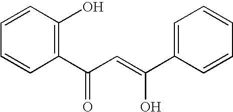 | 12.5 | 0.551 | 0.31 | 0.124 | 0.096 | 0.076 | 0.072 | 0.083 | 0.058 |
| 4'- HYDROXYCHALCONE | 1. Inhibition of superoxide anion and elastase release in human neutrophils by 3′-isopropoxy- chalcone via a cAMP- dependent pathway. British Journal of Pharmacology (2006), 148(1), 78- 87. 2. Metabolism of the a,b-unsaturated ketones, chalcone and trans-4- phenyl-3- buten-2-one, by rat liver microsornes and estrogenic activity of the metabolites. Drug Metabolism and Disposition (2005), 33(8), 1115-1123. | 11 | 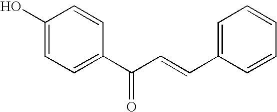 | 12.5 | 0.552 | 0.329 | 0.1 | 0.093 | 0.079 | 0.073 | 0.088 | 0.058 |
| 5- HYDROXYIMINOISOC ARYOPHYLLENE | | 12 | 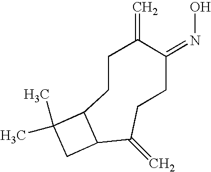 | 12.5 | 0.153 | 0.131 | 0.117 | 0.108 | 0.087 | 0.077 | 0.096 | 0.058 |
| CARYOPHYLLENYL ACETATE | | 12 | 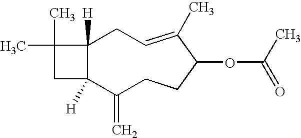 | 12.5 | 0.258 | 0.239 | 0.109 | 0.1 | 0.084 | 0.076 | 0.087 | 0.058 |
| PROPANIL | 1. Screening for estrogen and androgen receptor activities in 200 pesticides by in vitro reporter gene assays using chinese hamster ovary cells. Environmental Health Perspectives (2004), 112(5), 524-531. 2. Estrogenic activities of 517 chemicals by yeast two-hybrid assay. Journal of Health Science (2000), 46(4), 282-298. | 13 | 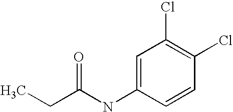 | 12.5 | 0.564 | 0.217 | 0.108 | 0.097 | 0.076 | 0.072 | 0.084 | 0.058 |
| IMPERATORIN | 1. Evaluation of the calcium antagonistic activity of Peucedanum ostruthium and Olea europaea constituents. Pharmaceutical and Pharmacological Letters (1991), 1(2), 78-81. | 17 | 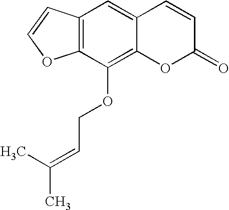 | 12.5 | 0.244 | 0.147 | 0.082 | 0.073 | 0.066 | 0.064 | 0.084 | 0.058 |
| 2,3,4-TRIHYDROXY-4'- ETHOXYBENZOPHEN ONE | | 19 | 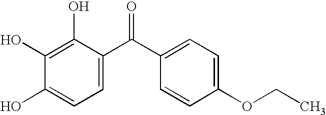 | 12.5 | 0.365 | 0.169 | 0.1 | 0.103 | 0.081 | 0.074 | 0.114 | 0.058 |
| DICLOFENAC SODIUM | It is an NSAID. | 21 | 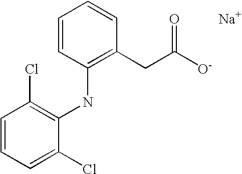 | 12.5 | 0.242 | 0.126 | 0.092 | 0.089 | 0.077 | 0.072 | 0.085 | 0.058 |
| NIMODIPINE | 1. Preparation of 4- [(benzimidazolyl/ pyrazolyl/ triazolyl) methoxy] phenoxyacetic acids as PPAR modulators. PCT Int. Appl. (2006), 62 pp. CODEN: PIXXD2 WO 2006084176 (many references to PPAR) | 22 | 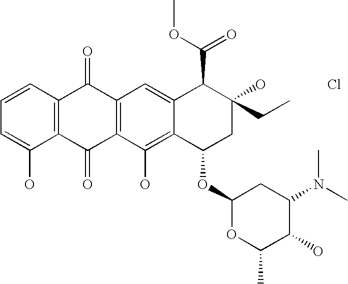 | 12.5 | 0.27 | 0.178 | 0.116 | 0.113 | 0.096 | 0.081 | 0.097 | 0.058 |
| AKLAVINE HYDROCHLORIDE | | | 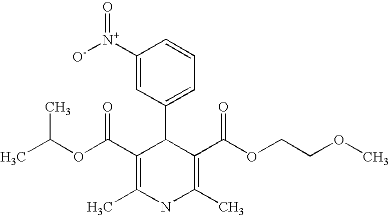 | 12.5 | 0.246 | 0.145 | 0.101 | 0.088 | 0.078 | 0.136 | 0.095 | 0.058 |
| ACETAMINOSALOL | 1. Identification of candidate drugs for the treatment of ALS. Amyotrophic Lateral Sclerosis and Other Motor Neuron Disorders (2005), 6(1), 29-36. | | 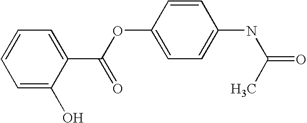 | 12.5 | 0.198 | 0.127 | 0.103 | 0.085 | 0.083 | 0.078 | 0.085 | 0.058 |
| 2-ISOPROPYL-3- METHOXYCINNAMIC ACID | | | 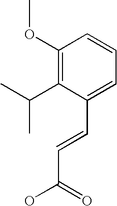 | 12.5 | 0.397 | 0.184 | 0.123 | 0.111 | 0.086 | 0.079 | 0.097 | 0.058 |
| SANGUINARINE SULFATE | | | 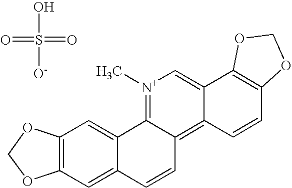 | 12.5 | 0.345 | 0.165 | 0.111 | 0.089 | 0.077 | 0.076 | 0.092 | 0.058 |
| CRASSIN ACETATE | 1. Cembranoid inhibitors of nicotinic acetylcholine receptors. PCT Int. Appl. (1998), 13 pp. CODEN: PIXXD2 WO 9855111 A1 19981210 | | 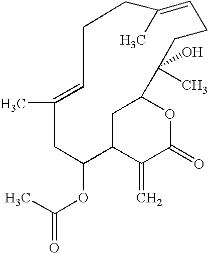 | 12.5 | 0.443 | 0.324 | 0.107 | 0.099 | 0.075 | 0.068 | 0.084 | 0.058 |
| DEHYDRO- PACHYRRHIZONE | | | 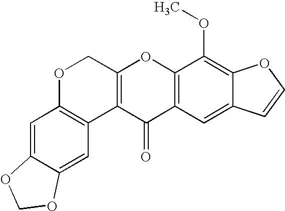 | 12.5 | 0.211 | 0.149 | 0.124 | 0.11 | 0.088 | 0.072 | 0.1 | 0.058 |
| THIAMYLAL SODIUM | | | 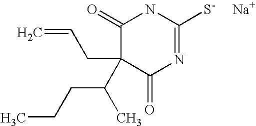 | 12.5 | 0.209 | 0.162 | 0.103 | 0.101 | 0.089 | 0.071 | 0.089 | 0.058 |
| DIURON | | 14 | 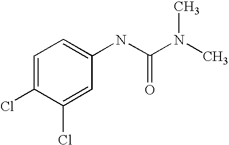 | 25 | 0.132 | 0.101 | 0.095 | 0.081 | 0.087 | 0.073 | 0.082 | 0.058 |
| HOMOPTEROCARPIN | | 16 | 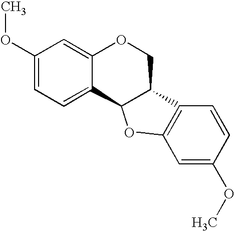 | 25 | 0.44 | 0.117 | 0.09 | 0.071 | 0.065 | 0.064 | 0.083 | 0.058 |
| MAACKIAIN | | 16 | 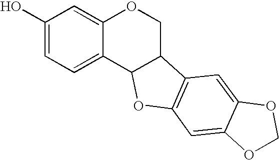 | 25 | 0.209 | 0.104 | 0.09 | 0.103 | 0.076 | 0.073 | 0.084 | 0.058 |
| PRENYLETIN | | 17 | 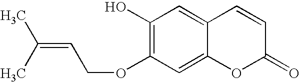 | 25 | 0.292 | 0.121 | 0.09 | 0.087 | 0.07 | 0.072 | 0.087 | 0.058 |
| (+)NAPROXEN | | 21 | 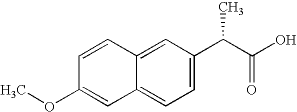 | 25 | 0.126 | 0.116 | 0.096 | 0.084 | 0.081 | 0.07 | 0.116 | 0.058 |
| DICTAMNINE | | | 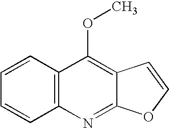 | 25 | 0.32 | 0.119 | 0.089 | 0.085 | 0.074 | 0.069 | 0.11 | 0.058 |
| METHYLORSELLINIC ACID, ETHYL ESTER | | | 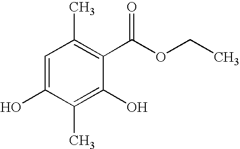 | 25 | 0.259 | 0.119 | 0.097 | 0.084 | 0.072 | 0.073 | 0.082 | 0.058 |
| LORATADINE | | | 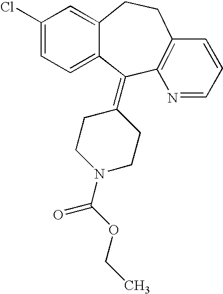 | 25 | 0.17 | 0.124 | 0.082 | 0.09 | 0.077 | 0.074 | 0.085 | 0.058 |
| CHLORPROPHAM | | | 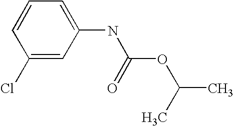 | 25 | 0.145 | 0.101 | 0.096 | 0.09 | 0.081 | 0.072 | 0.086 | 0.058 |
| 3,5-DIPRENYL-4- HYDROXYACETOPHE NONE | | | 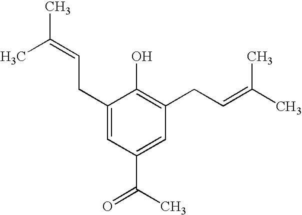 | 25 | 0.195 | 0.112 | 0.096 | 0.108 | 0.082 | 0.077 | 0.085 | 0.058 |
| 2-PROPYL-3- HYDROXYETHYLENE PYRAN-4-ONE | | | 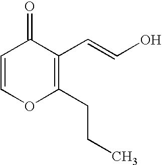 | 25 | 0.126 | 0.104 | 0.088 | 0.092 | 0.083 | 0.079 | 0.091 | 0.058 |

TABLE 3
Compounds That Restore Growth of pdr5 ypt1ts Cells

| | | 50 | 12.5 | 3.13 | 0 |
|---|---|---|---|---|---|
| Molecular Structure | Name | uM | uM | uM | uM |
| 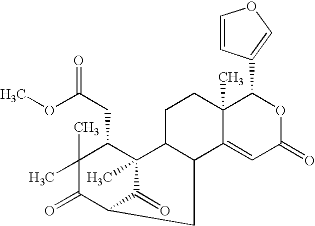 | CARAPIN | 0.5 | 0.357 | 0.22 | 0.077 |
| 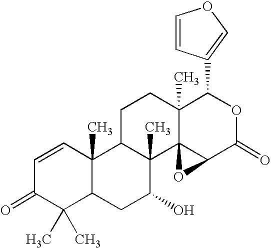 | DEACETYL- GEDUNIN | 0.377 | 0.185 | 0.116 | 0.077 |
| 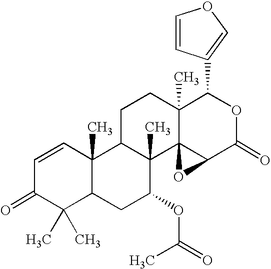 | GEDUNIN | 0.183 | 0.231 | 0.109 | 0.077 |
| 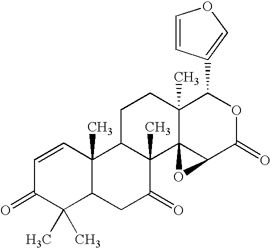 | DEACETOXY- 7-OXOGEDUNIN | 0.642 | 0.63 | 0.594 | 0.077 |
| 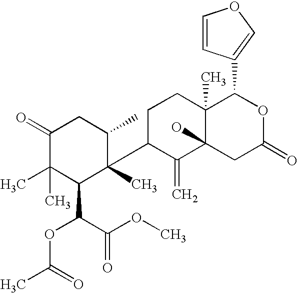 | 6-ACETOXY- ANGOLENSIC ACID METHYL ESTER | 0.489 | 0.364 | 0.131 | 0.077 |
| 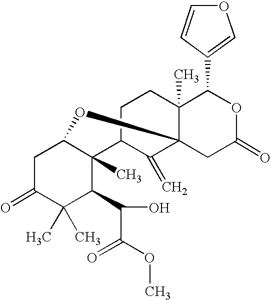 | 6-HYDROXY- ANGOLENSIC ACID METHYL ESTER | 0.626 | 0.561 | 0.232 | 0.077 |
| 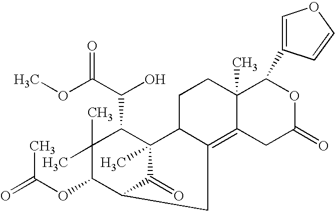 | SWIETENOLIDE- 3-ACETATE | 0.619 | 0.629 | 0.425 | 0.077 |
| 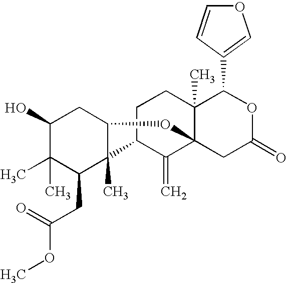 | 3-DEOXY-3beta- HYDROXY- ANGOLENSIC ACID METHYL ESTER | 0.621 | 0.641 | 0.384 | 0.077 |
| 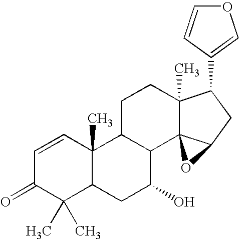 | TRI- CHILENONE | 0.462 | 0.283 | 0.119 | 0.077 |
| 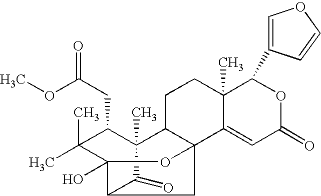 | 8beta- HYDROXY- CARAPIN, 3,8- HEMIACETAL | 0.486 | 0.383 | 0.151 | 0.077 |
| 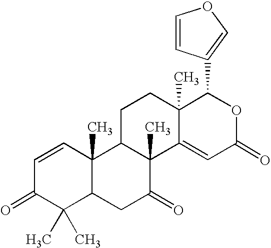 | 7-DEACETOXY- 7-OXODEOXY- GEDUNIN | 0.67 | 0.604 | 0.335 | 0.077 |
| 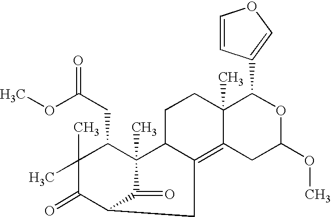 | 16-DEOXO- MEXICANOLIDE 16-METHYL ETHER | 0.55 | 0.528 | 0.3 | 0.077 |
| 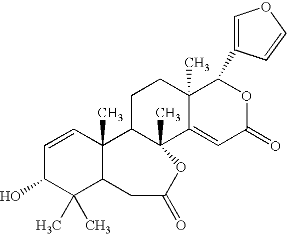 | DEOXY- ANDIROBIN LACTONE | 0.642 | 0.678 | 0.723 | 0.077 |
| 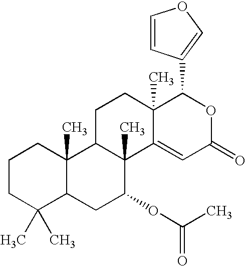 | DEOXO- DEOXY- DIHYDRO- GEDUNIN | 0.452 | 0.479 | 0.245 | 0.077 |
| 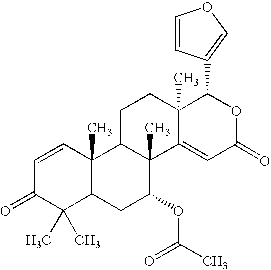 | DEOXY- GEDUNIN | 0.328 | 0.141 | 0.092 | 0.077 |
| 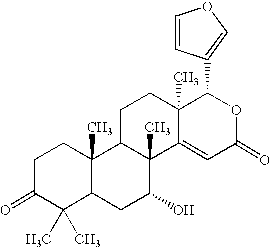 | DIHYDRO- 7- DESACETYLDE- OXYGEDUNIN | 0.328 | 0.568 | 0.197 | 0.077 |
| 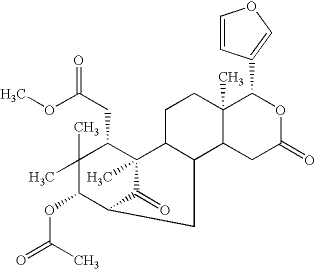 | DIHYDRO- FISSINOLIDE | 0.308 | 0.085 | 0.079 | 0.077 |
| 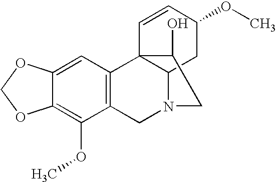 | AMBELLINE | 0.057 | 0.302 | 0.071 | 0.077 |
| 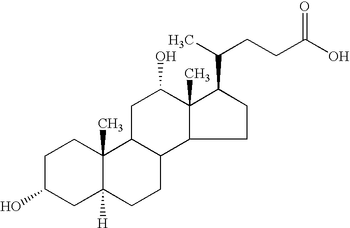 | DEOXY- CHOLIC ACID | 0.133 | 0.481 | 0.233 | 0.077 |
| 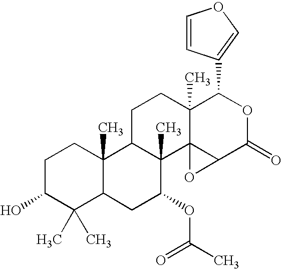 | 3alpha- HYDROXY- DEOXO- DIHYDRO- GEDUNIN | 0.146 | 0.25 | 0.137 | 0.077 |
| 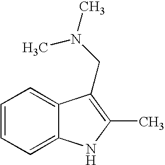 | 2-METHYL GRAMINE | 0.295 | 0.106 | 0.084 | 0.077 |
| 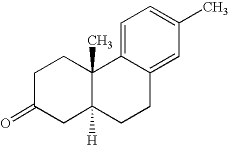 | 13-METHYL- 4,4-BISNOR- 8,11,13- PODO- CARPATRIEN- 3-ONE | 0.424 | 0.152 | 0.082 | 0.077 |
| 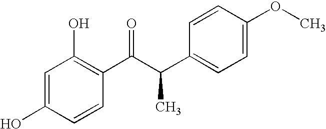 | ANGOLENSIN (R) | 0.062 | 0.652 | 0.327 | 0.077 |
| 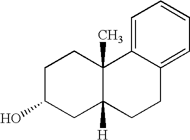 | 3alpha- HYDROXY-4,4- BISNOR- 8,11,13- PODO- CARPATRIENE | 0.408 | 0.104 | 0.074 | 0.077 |
| 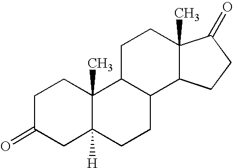 | 5alpha- ANDROSTAN- 3,17-DIONE | 0.319 | 0.16 | 0.11 | 0.077 |
| 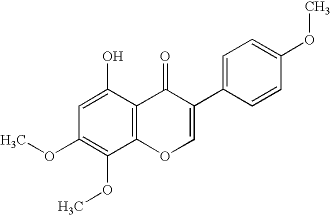 | ISO- TECTORIGENIN, 7-METHYL ETHER | 0.595 | 0.147 | 0.09 | 0.077 |
| 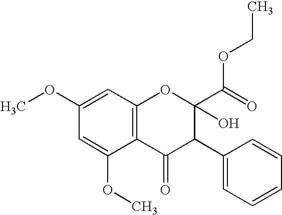 | 2-ETHOXY- CARBONYL-2- HYDROXY-5,7- DIMETHOXY- ISOFLAVANONE | 0.468 | 0.134 | 0.091 | 0.077 |
| 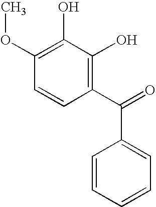 | 3-DESMETHYL- 5-DES- HYDROXY- SCLEROIN | 0.556 | 0.149 | 0.11 | 0.077 |
| 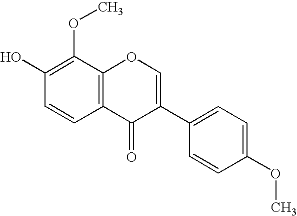 | 7-HYDROXY- 8,4′-DIMETH- OXYISO- FLAVONE | 0.398 | 0.151 | 0.121 | 0.077 |
| 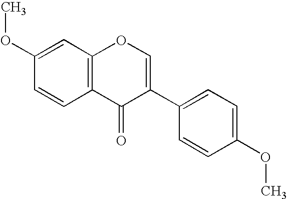 | 7,4′- DIMETH- OXYISO- FLAVONE | 0.513 | 0.551 | 0.222 | 0.077 |
| 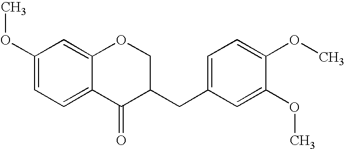 | DEOXY- SAPPANONE B TRIMETHYL ETHER | 0.514 | 0.203 | 0.107 | 0.077 |
| 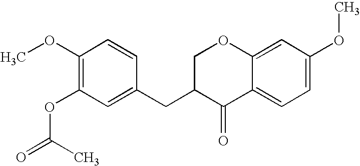 | DEOXY- SAPPANONE B 7,3′-DI- METHYL ETHER ACETATE | 0.374 | 0.17 | 0.137 | 0.077 |
| 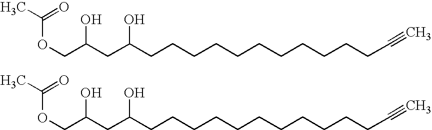 | AVOCATIN A | 0.036 | 0.06 | 0.351 | 0.077 |
| 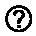 | CITRININ | 0.38 | 0.158 | 0.102 | 0.077 |
| 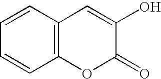 | 3- HYDROXY- COUMARIN | 0.299 | 0.109 | 0.083 | 0.077 |
| 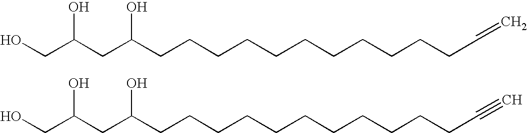 | AVOCATIN B | 0.032 | 0.032 | 0.451 | 0.077 |
| 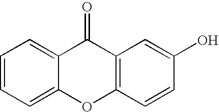 | 2-HYDROXY- XANTHONE | 0.571 | 0.257 | 0.107 | 0.077 |
| 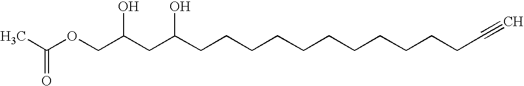 | AVOCADYNE ACETATE | 0.039 | 0.406 | 0.389 | 0.077 |
| 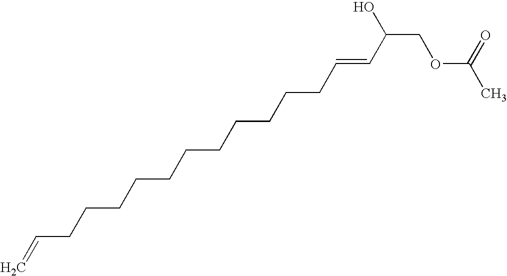 | DEHYDRO- AVOCATIN - 1-ACETATE | 0.365 | 0.329 | 0.116 | 0.077 |
| 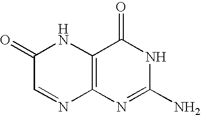 | XANTHOP- TERIN | 0.073 | 0.23 | 0.101 | 0.077 |
| 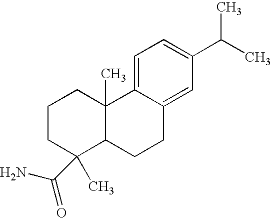 | DEHYDRO- ABIETAMIDE | 0.041 | 0.419 | 0.272 | 0.077 |
| 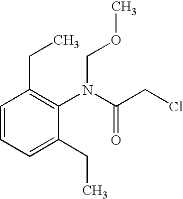 | ALACHLOR | 0.45 | 0.142 | 0.101 | 0.077 |
| 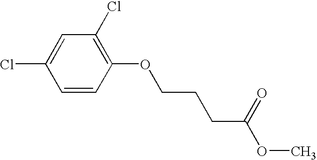 | 2,4-DICHLORO- PHENOXY- BUTYRIC ACID, METHYL ESTER | 0.221 | 0.255 | 0.159 | 0.077 |
| 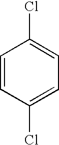 | PARA- DICHLORO- BENZENE | 0.202 | 0.28 | 0.169 | 0.077 |
| 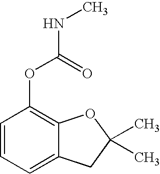 | CARBO- FURAN | 0.239 | 0.264 | 0.176 | 0.077 |
| 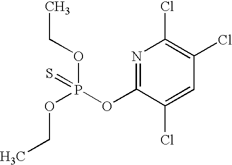 | CHLOR- PYRIFOS | 0.253 | 0.166 | 0.122 | 0.077 |
| 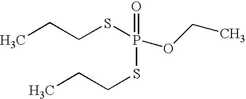 | ETHOPROP | 0.251 | 0.134 | 0.118 | 0.077 |
| 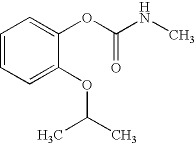 | PROPOXUR | 0.231 | 0.289 | 0.194 | 0.077 |
| 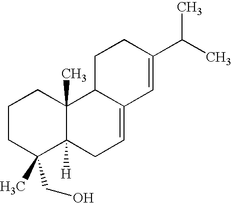 | ABIETANOL | 0.29 | 0.274 | 0.102 | 0.077 |
| 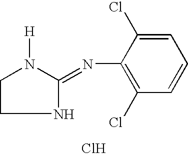 | CLONIDINE HYDRO- CHLORIDE | 0.064 | 0.095 | 0.237 | 0.077 |
| 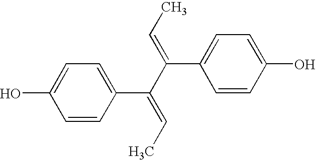 | DIENESTROL | 0.044 | 0.309 | 0.142 | 0.077 |
| 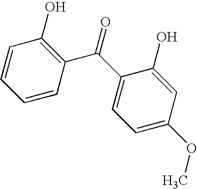 | DIOXY- BENZONE | 0.579 | 0.12 | 0.088 | 0.077 |
| 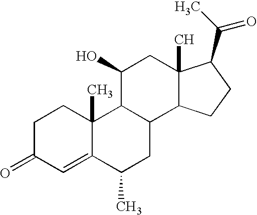 | MEDRY- SONE | 0.266 | 0.165 | 0.114 | 0.077 |
| 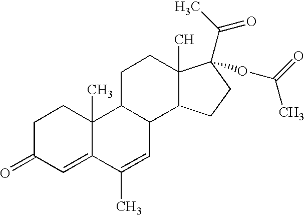 | MEGESTROL ACETATE | 0.202 | 0.341 | 0.222 | 0.077 |
| 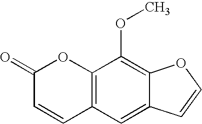 | METHOX- SALEN | 0.38 | 0.25 | 0.124 | 0.077 |
| 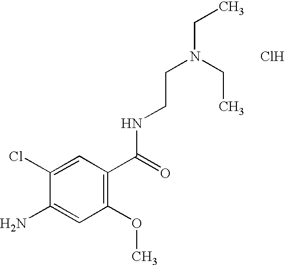 | METO- CLOPRAMIDE HYDRO- CHLORIDE | 0.555 | 0.158 | 0.099 | 0.077 |
| 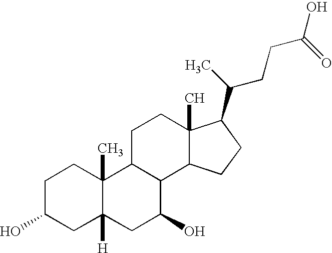 | URSODIOL | 0.523 | 0.41 | 0.202 | 0.077 |
| 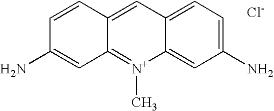 | ACRIFLAV- INIUM HYDRO- CHLORIDE | 0.151 | 0.229 | 0.126 | 0.077 |
| 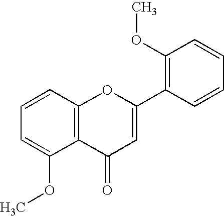 | 5,2′- DIMETHOXY- FLAVONE | 0.383 | 0.256 | 0.088 | 0.077 |
| 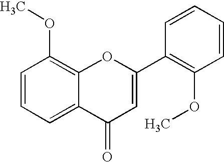 | 8,2′- DIMETHOXY- FLAVONE | 0.674 | 0.234 | 0.083 | 0.077 |
| 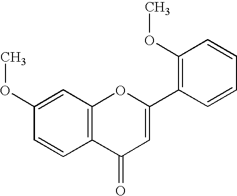 | 7,2′- DIMETHOXY- FLAVONE | 0.3 | 0.156 | 0.098 | 0.077 |
| 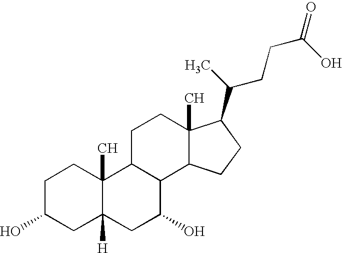 | CHENODIOL | 0.15 | 0.285 | 0.126 | 0.077 |
| 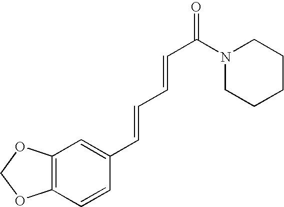 | PIPERINE | 0.377 | 0.135 | 0.107 | 0.077 |
| 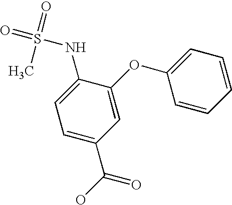 | NIMESULIDE | 0.539 | 0.147 | 0.098 | 0.077 |
| 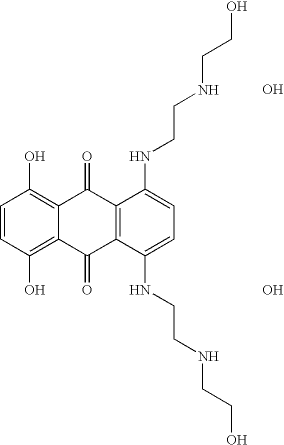 | MITO- XANTHRONE HYDRO- CHLORIDE | 0.313 | 0.132 | 0.149 | 0.077 |
| 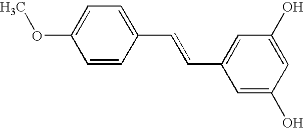 | RESVERA- TROL 4′- METHYL ETHER | 0.331 | 0.103 | 0.08 | 0.077 |
| 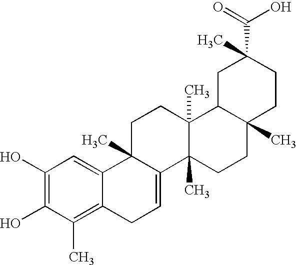 | DIHYDRO- CELASTROL | 0.33 | 0.526 | 0.119 | 0.077 |
| 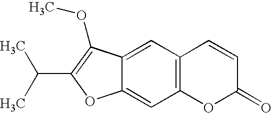 | PEUCEDANIN | 0.461 | 0.25 | 0.138 | 0.077 |
| 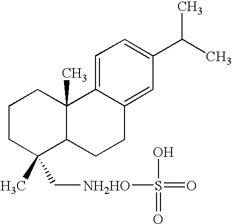 | 18-AMINO ABIETA- 8,11,13- TRIENE SULFATE | 0.378 | 0.06 | 0.055 | 0.077 |
| 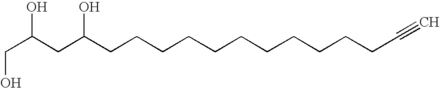 | AVO- CADYNE | 0.265 | 0.425 | 0.132 | 0.077 |
| 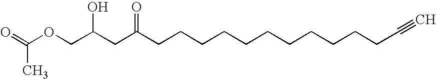 | AVO- CADYNONE ACETATE | 0.057 | 0.275 | 0.126 | 0.077 |
| 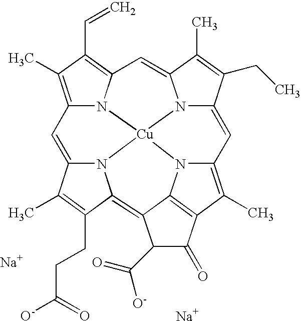 | CHLORO- PHYLLIDE Cu COMPLEX Na SALT | 0.305 | 0.054 | 0.064 | 0.077 |
| 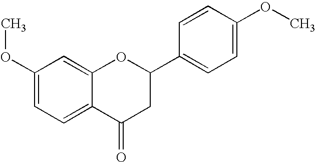 | LIQUIRITI- GENIN DIMETHYL ETHER | 0.364 | 0.13 | 0.086 | 0.077 |
| 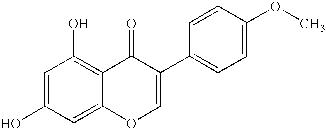 | BIO- CHANIN A | 0.055 | 0.297 | 0.112 | 0.077 |

What is claimed is:

1. A method of treating or preventing a disorder characterized by impaired protein trafficking, the method comprising administering to a subject in need thereof an effective amount of a compound of Table 2 or a pharmaceutically acceptable derivative thereof or Table 3 or a pharmaceutically acceptable derivative thereof.

2. The method of claim 1, wherein the disorder is a synucleinopathy.

3. The method of claim 2, wherein the synucleinopathy is Parkinson's disease, Lewy body disease, the Lewy body variant of Alzheimer's disease, dementia with Lewy bodies, multiple system atrophy, or the Parkinsonism-dementia complex of Guam.

4. The method of claim 1, wherein the disorder is a lysosomal storage disorder.

5. The method of claim 4, wherein the lysosomal storage disorder is Fabry disease, Farber disease, Gaucher disease, GM1-gangliosidosis, Tay-Sachs disease, Sandhoff disease, GM2 activator disease, Krabbe disease, metachromatic leukodystrophy, Niemann-Pick disease (types A, B, and C), Hurler disease, Scheie disease, Hunter disease, Sanfilippo disease, Morquio disease, Maroteaux-Lamy disease, hyaluronidase deficiency, aspartylglucosaminuria, fucosidosis, mannosidosis, Schindler disease, sialidosis type 1, Pompe disease, Pycnodysostosis, ceroid lipofuscinosis, cholesterol ester storage disease, Wolman disease, Multiple sulfatase, galactosialidosis, mucolipidosis (types II, III, and IV), cystinosis, sialic acid storage disorder, chylomicron retention disease with Marinesco-Sjögren syndrome, Hermansky-Pudlak syndrome, Chediak-Higashi syndrome, Danon disease, or Geleophysic dysplasia.

6. The method of claim 1, wherein the disorder is characterized by an impaired delivery of cargo to a cellular compartment.

7. The method of claim 1, wherein the disorder is characterized by a Rab27a mutation or a deficiency of Rab27a.

8. The method of claim 7, wherein the disorder is Griscelli syndrome.

9. The method of claim 1, wherein the disorder is cystic fibrosis.

10. The method of claim 1, wherein the disorder is diabetes.

11. The method of claim 10, wherein the diabetes is diabetes mellitus.

12. The method of claim 1, wherein the disorder is hereditary emphysema, α-1 antitrypsin deficiency, hereditary hemochromatosis, oculocutaneous albinism, protein C deficiency, type I hereditary angioedema, congenital sucrase-isomaltase deficiency, Crigler-Najjar type II, Laron syndrome, hereditary Myeloperoxidase, primary hypothyroidism, congenital long QT syndrome, tyroxine binding globulin deficiency, familial hypercholesterolemia, familial chylomicronemia, abeta-lipoproteinema, low plasma lipoprotein a levels, hereditary emphysema with liver injury, congenital hypothyroidism, osteogenesis imperfecta, hereditary hypofibrinogenemia, alpha-1antichymotrypsin deficiency, nephrogenic diabetes insipidus, neurohypophyseal diabetes, insipidus, Charcot-Marie-Tooth syndrome, Pelizaeus Merzbacher disease, von Willebrand disease type IIA, combined factors V and VIII deficiency, spondylo-epiphyseal dysplasia tarda, choroideremia, I cell disease, Batten disease, ataxia telangiectasias, acute lymphoblastic leukemia, acute myeloid leukemia, myeloid leukemia, ADPKD-autosomal dominant polycystic kidney disease, microvillus inclusion disease, tuberous sclerosis, oculocerebro-renal syndrome of Lowe, amyotrophic lateral sclerosis, myelodysplastic syndrome, Bare lymphocyte syndrome, Tangier disease, familial intrahepatic cholestasis, X-linked adreno-leukodystrophy, Scott syndrome, Hermansky-Pudlak syndrome types 1 and 2, Zellweger syndrome, rhizomelic chondrodysplasia puncta, autosomal recessive primary hyperoxaluria, Mohr Tranebjaerg syndrome, spinal and bullar muscular atrophy, primary ciliary diskenesia (Kartagener's syndrome), Miller Dieker syndrome, lissencephaly, motor neuron disease, Usher's syndrome, Wiskott-Aldrich syndrome, Optiz syndrome, Huntington's disease, hereditary pancreatitis, anti-phospholipid syndrome, overlap connective tissue disease, Sjögren's syndrome, stiff-man syndrome, Brugada syndrome, congenital nephritic syndrome of the Finnish type, Dubin-Johnson syndrome, X-linked hypophosphosphatemia, Pendred syndrome, persistent hyperinsulinemic hypoglycemia of infancy, hereditary spherocytosis, aceruloplasminemia, infantile neuronal ceroid lipofuscinosis, pseudoachondroplasia and multiple epiphyseal, Stargardt-like macular dystrophy, X-linked Charcot-Marie-Tooth disease, autosomal dominant retinitis pigmentosa, Wolcott-Rallison syndrome, Cushing's disease, limb-girdle muscular dystrophy, mucoploy-saccharidosis type IV, hereditary familial amyloidosis of Finish, Anderson disease, sarcoma, chronic myelomonocytic leukemia, cardiomyopathy, faciogenital dysplasia, Torsion disease, Huntington and spinocerebellar ataxias, hereditary hyperhomosyteinemia, polyneuropathy, lower motor neuron disease, pigmented retinitis, seronegative polyarthritis, interstitial pulmonary fibrosis, Raynaud's phenomenon, Wegner's granulomatosis, preoteinuria, CDG-Ia, CDG-Ib, CDG-Ic, CDG-Id, CDG-Ie, CDG-If, CDG-IIa, CDG-IIb, CDG-IIc, CDG-IId, Ehlers-Danlos syndrome, multiple exostoses, Griscelli syndrome (type 1 or type 2), or X-linked non-specific mental retardation.

13. The method of claim 1, wherein the subject is a human.

14. A composition comprising (i) a compound of Table 2 or a pharmaceutically acceptable derivative thereof or Table 3 or a pharmaceutically acceptable derivative thereof, and (ii) one or more of donepezil hydrochloride (Aracept), rivastigmine tartrate (Exelon), tacrine hydrochloride (Cognex), or galantamine hydrobromide (Reminyl).

15. A method of treating or preventing a synucleinopathy, the method comprising administering to a subject in need thereof an effective amount of the composition of claim 14.

16. The method of claim 15, wherein the synucleinopathy is Parkinson's disease, Lewy body disease, the Lewy body variant of Alzheimer's disease, dementia with Lewy bodies, multiple system atrophy, or the Parkinsonism-dementia complex of Guam.

17. The method of claim 15, wherein the subject is a human.

18. A method of inhibiting alpha synuclein-mediated cellular toxicity, the method comprising contacting a cell expressing a toxicity-inducing amount or form of alpha synuclein with an effective amount of a compound of Table 2 or a pharmaceutically acceptable derivative thereof or Table 3 or a pharmaceutically acceptable derivative thereof.

19. A method of inhibiting alpha synuclein-mediated cellular toxicity, the method comprising contacting a cell expressing a toxicity-inducing amount or form of alpha synuclein with an effective amount of the composition of claim 15.

20. A method of producing a protein, the method comprising:
culturing a cell in the presence of a compound of Table 2 or a pharmaceutically acceptable derivative thereof or Table 3 or a pharmaceutically acceptable derivative thereof; and
purifying a protein produced by the cell,
wherein the culturing of the cell in the presence of the compound results in enhanced production of the purified protein as compared to culture of the cell in the absence of the compound.

21. The method of claim 20, wherein the protein is a recombinant protein encoded by a heterologous nucleic acid.

22. The method of claim 20, wherein the protein is a secreted protein

23. The method of claim 20, wherein the protein is a glycosylated protein.

24. The method of claim 20, wherein the protein is a cytokine, a lymphokine, a growth factor, or an antibody.

25. The method of claim 20, wherein the cell is an insect cell, a mammalian cell, a fungal cell, or a bacterial cell.

26. The method of claim 25, wherein the cell is a Chinese Hamster Ovary (CHO) cell.
